(19) United States
(12) Patent Application Publication
(10) Pub. No.: US 2025/0049393 A1
(43) Pub. Date: Feb. 13, 2025

(54) METHOD AND SYSTEM FOR A SMART MASK

(71) Applicant: Northwestern University, Evanston, IL (US)

(72) Inventors: Josiah David Hester, Evanston, IL (US); Alexander Curtiss, Chicago, IL (US); Blaine Rothrock, Chicago, IL (US); Nabil Iyad Alshurafa, Chicago, IL (US)

(21) Appl. No.: 18/718,693

(22) PCT Filed: Dec. 13, 2022
(86) PCT No.: PCT/US2022/081418

§ 371 Date: Jun. 11, 2024

Related U.S. Application Data
(60) Provisional application No. 63/288,968, filed on Dec. 13, 2021.

(51) Int. Cl. A61B 5/00; A41D 13/11; A61B 5/0205

(52) U.S. Cl. CPC A61B 5/6803; A41D 13/11; A61B 5/02055; A61B 2562/0219

(57) ABSTRACT

A smart face mask device includes a housing that is designed to mount to a personal protective face mask and a memory within the housing that is configured to store sensor data. The device includes one or more sensors configured to generate the sensor data during use of the personal protective mask. The device also includes a processor operatively coupled to the memory and configured to process the sensor data to determine information regarding a user of the personal protective mask, and to transmit the information regarding the user to a remote application.

### CROSS-REFERENCE TO RELATED APPLICATION

The present application claims the priority benefit of U.S. Provisional Patent App. No. 63/288,968 filed on Dec. 13, 2021, the entire disclosure of which is incorporated by reference herein.

### STATEMENT REGARDING FEDERALLY SPONSORED RESEARCH

This invention was made with government support under CNS2032408 awarded by the National Science Foundation. The government has certain rights in the invention.

### BACKGROUND

Due to the COVID-19 pandemic, face masks have become ubiquitous worldwide. As a result of the pandemic and due to the incredibly infectious nature of COVID-19 and its variants (e.g., the B.1.617.2 (Delta) variant), the U.S. Centers for Disease Control and Prevention (CDC) and the World Health Organization (WHO) have recommended nearly universal masking at times, with many governments enforcing mask mandates. Because of the slow rate of global vaccination and the continual spread of the virus, it appears that COVID-19 will remain a threat for years to come. While the pandemic has evolved and public health messaging with it, the general consensus is that, beyond a vaccination, masking is one of the best forms of protection.

### SUMMARY

An illustrative smart face mask device includes a housing that is designed to mount to a personal protective face mask and a memory within the housing that is configured to store sensor data. The device includes one or more sensors configured to generate the sensor data during use of the personal protective mask. The device also includes a processor operatively coupled to the memory and configured to process the sensor data to determine information regarding a user of the personal protective mask, and to transmit the information regarding the user to a remote application.

In an illustrative embodiment, the one or more sensors include a pressure sensor such that the sensor data includes sensed pressure values within the personal protective face mask. In such an embodiment, the processor is configured to process the sensed pressure values to assess mask fit of the personal protective face mask. Specifically, the processor is configured to determine an integral of a pressure drop across the personal protective face mask to assess the mask fit. The processor is also configured to generate an alert responsive to a determination that the mask fit of the personal protective face mask does not satisfy a mask fit threshold.

In another embodiment, the one or more sensors include a temperature sensor such that the sensor data includes temperature values within the personal protective face mask. In such an embodiment, the processor processes the temperature values to determine a respiration rate of the user of the personal protective face mask. The processor also generates an alert responsive to a determination that the respiration rate is not within a threshold range of respiration rates. In another embodiment, the one or more sensors include an inertial measurement unit such that the sensor data includes information regarding movement of the personal protective face mask. In such an embodiment, the processor processes the information regarding movement of the personal protective face mask to determine a heartbeat of the user of the personal protective face mask. The processor also generates an alert responsive to a determination that the heartbeat of the user falls outside of a threshold range of heartbeats.

In another embodiment, the one or more sensors include a pressure sensor, and the processor is configured to determine whether the mask is being worn based on pressure readings from the pressure sensor. In one embodiment, the processor is configured to determine an amount of time that the mask has been worn based on the pressure readings from the pressure sensor. In another embodiment, the processor is configured to generate an alert to change masks responsive to a determination that the amount of time that the mask has been worn exceeds a threshold value. In another illustrative embodiment, a first magnet is mounted to the housing. The first magnet is configured to mate with a second magnet on the personal protective face mask to secure the device to the personal protective face mask.

An illustrative method of monitoring a face mask includes obtaining sensor data from one or more sensors mounted to a housing that is secured to a personal protective face mask. The method also includes storing the sensor data in a memory that is within the housing. The method also includes processing, by a processor operatively coupled to the memory, the sensor data to determine information regarding a user of the personal protective mask. The method further includes transmitting the information regarding the user to a remote application.

In one embodiment, the one or more sensors include a pressure sensor such that obtaining the sensor data includes obtaining pressure values within the personal protective face mask. The method can also include processing, by the processor, the obtained pressure values to assess mask fit of the personal protective face mask. In another embodiment, the one or more sensors include a temperature sensor such that obtaining the sensor data includes obtaining temperature values within the personal protective face mask. The method can further include processing, by the processor, the obtained temperature values to determine a respiration rate of the user of the personal protective face mask.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts an overview of the smart mask platform and concept in accordance with an illustrative embodiment.

FIG. 2 is a diagram that shows survey results regarding the relative importance of various system metrics in accordance with an illustrative embodiment.

FIG. 3A depicts various energy harvesters (shaker, TEG, flexible solar panel, and SATURN) and their dimensions in accordance with an illustrative embodiment.

FIG. 3B depicts power harvested from activities with different harvesters along with mask placement in accordance with an illustrative embodiment.

FIG. 4 is a block diagram overview of the FaceBit hardware in accordance with an illustrative embodiment.

FIG. 5A depicts a top view of a circuit board for the proposed system in accordance with an illustrative embodiment.

FIG. 5B depicts a bottom view of the circuit board for the proposed system in accordance with an illustrative embodiment.

FIG. 5C depicts an internal view of the circuit board (FaceBit) mounted to a mask in accordance with an illustrative embodiment.

FIG. 5D depicts an external view of the mask with a magnet used to secure the circuit board (FaceBit) in accordance with an illustrative embodiment.

FIG. 6 depicts a hybrid power architecture of the FaceBit platform in accordance with an illustrative embodiment.

FIG. 7 is an overview of the energy-efficient runtime of the smart mask system in accordance with an illustrative embodiment.

FIG. 8 depicts an example pressure signal which follows a user through a normal sequence of activities in accordance with an illustrative embodiment.

FIG. 9A shows a recovered BCG/heartbeat signal obtained from a seated participant wearing an N95 mask fitted with the FaceBit, along with an ECG signal recorded at the same time in accordance with an illustrative embodiment.

FIG. 9B shows the signal obtained from FaceBit's temperature sensor and pressure during tidal breathing in an N95 Mask in accordance with an illustrative embodiment.

FIG. 9C depicts cumulative integration of the pressure signal sensed inside an N95 mask while under suction from a 12V vacuum pump in accordance with an illustrative embodiment.

FIG. 10 shows a signal processing pipeline for the proposed system in accordance with an illustrative embodiment.

FIG. 11 shows an example raw signal and the output of the respiratory monitoring algorithm in accordance with an illustrative embodiment.

FIG. 12A depicts that FaceBit phone application homepage displaying general details, current wear time, respiration rate, heart-rate, and temperature data from the FaceBit sensor board in accordance with an illustrative embodiment.

FIG. 12B depicts the mask wear time interface for tracking mask disposal in accordance with an illustrative embodiment.

FIG. 12C is the FaceBit sensor board detail screen outlining current connection status in accordance with an illustrative embodiment.

FIG. 13A is a box-plot of each participant in the respiratory rate evaluation, highlighting number of samples and outliers in accordance with an illustrative embodiment.

FIG. 13B is a Bland-Altman plot comparing FaceBit's error to ground truth, and showing a tendency to report slightly above the ground truth and to be less accurate with higher ground truth reports in accordance with an illustrative embodiment.

FIG. 13C shows the cumulative distribution function over the evaluation samples in accordance with an illustrative embodiment.

FIG. 14A is a box-plot of each participant in the heart rate evaluation, highlighting samples per participant and outliers in accordance with an illustrative embodiment.

FIG. 14B is a Bland-Altman plot showing FaceBit reporting better accuracy in the lower heart rate ranges in accordance with an illustrative embodiment.

FIG. 14C shows probability and cumulative distribution function over the evaluation samples in accordance with an illustrative embodiment.

FIG. 15A depicts signal from inspiratory vital capacity tests (IVCT) recorded on-board FaceBit showing filtered pressure signals for each participant (n=3) grouped by fit in accordance with an illustrative embodiment.

FIG. 15B shows integral values from initial evaluation for participants and each experiment compared to each leak configuration in accordance with an illustrative embodiment.

FIG. 16 depicts results of the different mask-fit configurations in accordance with an illustrative embodiment.

FIG. 17 depicts results from the walk to lunch scenario in accordance with an illustrative embodiment.

FIG. 18 shows the FaceBit reported respiration rate plotted over the calculated rolling average of instantaneous respiration rates from the ground truth respiration belt in accordance with an illustrative embodiment.

FIG. 19 depicts thermistor placements in a N95 mask in accordance with an illustrative embodiment.

FIG. 20 shows filtered results of the thermistor analysis in accordance with an illustrative embodiment.

FIG. 21 is a table that includes a summary of the power and energy requirements of the various states that comprise the FaceBit application in accordance with an illustrative embodiment.

FIG. 22 depicts an application power trace over time while calculating health metrics in accordance with an illustrative embodiment.

FIG. 23 is a block diagram of components included in a smart mask system in accordance with an illustrative embodiment.

### DETAILED DESCRIPTION

The COVID-19 pandemic has dramatically increased the use of face masks across the world. Aside from physical distancing, they are among the most effective protection for healthcare workers and the general population. Traditional face masks are passive devices, however, and cannot alert the user in case of improper fit or mask degradation. Additionally, the inventors have recognized that face masks are optimally positioned to give unique insight into personal health metrics of the wearer. Recognizing this limitation and opportunity, described herein is FaceBit, which is an open-source platform for smart face mask applications. FaceBit's design was informed by need-finding studies with a cohort of health professionals. Small and easily secured into any face mask, FaceBit is accompanied by a mobile application that provides a user interface and facilitates research.

The FaceBit system includes software and hardware that monitors heart rate without skin contact via ballistocardiography, respiration rate via temperature changes, and mask-fit and wear time from pressure signals, all on-device with an energy-efficient runtime system. In one embodiment, FaceBit can harvest energy from breathing, motion, or sunlight to supplement a primary cell battery that alone delivers a battery lifetime of 11 days or more. Alternatively, the FaceBit may include a larger battery and not use energy harvesting techniques. FaceBit empowers the mobile computing community to jumpstart research in smart face mask sensing and inference, and provides a sustainable, convenient form factor for health management, applicable to COVID-19 frontline workers and beyond.

In light of the pandemic, the longstanding limits of traditional personal protective equipment (PPE) such as face masks have become apparent. First, face masks must be managed; many types, like N95s, must be professionally managed. This requires healthcare workers to take time away from caring for their patients. Moreover, traditional PPE offers only passive, as opposed to active protection. Finally, today's face masks miss opportunities for sensing and intelligence that could help to inform and actively protect wearers. Instrumented face masks are exceptionally well placed to provide active protection for frontline health workers and beyond because they are in (necessarily) close proximity to the respiratory system. The respiratory system is the target of many infectious diseases (including COVID-19), and also tightly coupled to general health.

Active personal protective equipment (PPE) in the form of “smart” face masks provide an early warning system when health or environmental metrics indicate danger; such as heart rate, respiratory rate (which is currently monitored via a wearable setting), or toxic air pollution. A wearer could be informed immediately if a respirator mask was not fitted correctly or if a leak formed during activity, as workers cannot always detect if a respirator mask is fitting loosely because of numbness of the face, fatigue, and mask wear and tear. If a mask had been worn too long, the active PPE system can notify the wearer. Finally, if a user is able to rely on a mask they already must wear to function as a wearable health tracker, this would provide better usability and adherence advantage. These use cases represent only the start of research questions in the space. With the rapidly changing pandemic world, it is imperative that the mobile computing and health communities have the tools to research the issues that relate to current challenges.

Realizing the above-discussed vision of smart face masks is challenging due to considerations in cost, size, weight, maintenance requirements, etc. Bulky, technologically complex, and power intensive face masks do not scale to the needs of a global pandemic. Rather, masks must be available and ready to use anytime, they must be either cheap and disposable, or sturdy to repeated use, decontamination, and wear. Masks are constantly replaced in clinical settings, necessitating a mechanism to transfer intelligent sensing capability from one mask to the other, easily and quickly. Masks are small and lightweight, meaning that any extra weight is noticeable and burdensome.

The inventors navigated these concerns and tradeoffs and develop FaceBit, a low cost, low weight, energy-efficient, energy harvesting, rich sensing platform. FaceBit is also a research platform, and its efficacy and general potential have been demonstrated by gathering information about its accuracy and usability. FaceBit is designed to be used in existing masks without modification and is easily temporarily clipped onto N95, and other surgical masks with a small magnet.

FaceBit is a modular hardware platform capable of supplementing a small primary cell battery with energy harvested from movement, sunlight, and even breathing. This combination extends the lifetime and reduces battery replacement frequency, but does not sacrifice size and burden. In an alternative embodiment, a larger capacity battery may be used such that the system does not include energy harvesting hardware/software. FaceBit collects and analyzes data from a suite of on-device sensors, and wirelessly sends heart rate, respiration rate, mask on/off status and other metrics to a custom phone application which allows for visualization, data analysis, and can also serve as an intervention system.

Specifically, after a series of design research studies on energy harvesting capability and need—with healthcare workers, the inventors designed a hardware and software platform that includes the FaceBit circuit board and a custom phone application that supports data collection, visualization, and alerts. The inventors studied sensing targets obtainable from a mask in a series of lab experiments and free-living scenarios, and designed a low-memory footprint, on-device signal processing pipeline to capture heart rate from subtle head movements, respiration rate from temperature changes in-mask, and explore leak detection via changes in in-mask pressure. To ensure long battery lifetime and low user-burden, an energy-efficient runtime and triggering architecture was designed for on-device capture of heart rate, respiratory rate, and mask wear time.

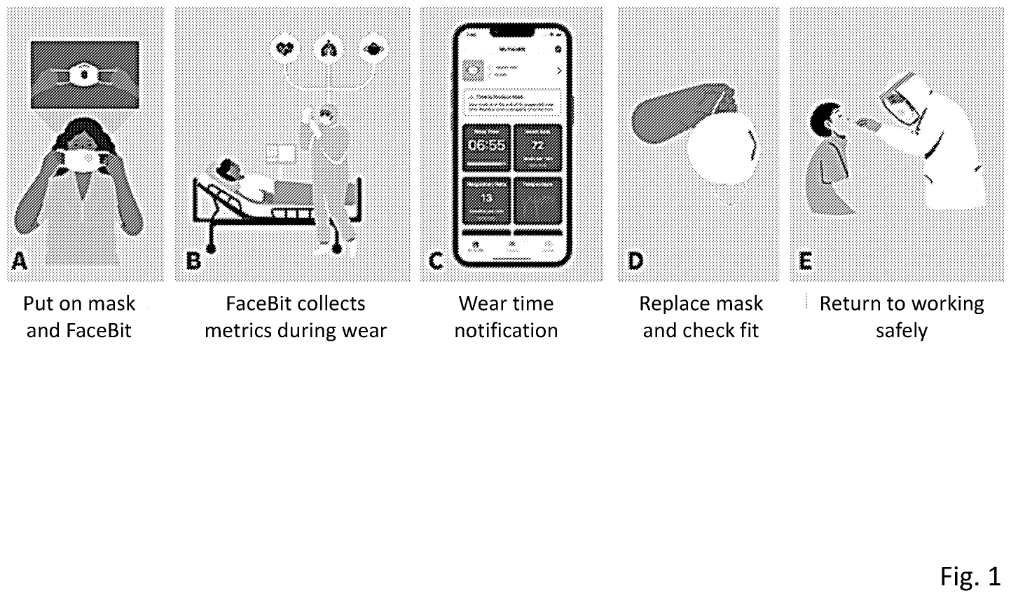

FIG. 1 depicts an overview of the smart mask platform and concept in accordance with an illustrative embodiment. Initially, the user mounts a FaceBit to a mask and puts on the mask. The FaceBit in the mask collects metrics during wear such as health and safety metrics like heart rate, respiratory rate, and wear time. Notifications regarding the monitored metrics are sent to a phone application (or other computing device) upon detection of state changes or other thresholds. Upon receiving such a notification that the mask should be replaced, the user replaces the mask, remounts the FaceBit into the new mask, and returns to working safely.

The proposed system builds on and expands various existing technologies to result in a cost-effective efficient device. As discussed, in some embodiments, the proposed system can use energy harvesting to enable long battery life. Many devices have adopted energy harvesting techniques to extend lifetimes and reduce maintenance without increasing bulk by adding large batteries. Early work, such as Trio, and Prometheus, deployed devices with solar panels for long term infrastructure and environmental monitoring. Permamote is a device that makes use of a hybrid energy solution, with a primary cell as backup for consistent readings, and energy harvester to extend lifetime. FaceBit extends beyond Permamote in leveraging more predictable energy modalities based on the mask being worn (i.e. breathing, motion). Other work has explored novel energy harvesting scenarios to power interactive devices, like SATURN and Paper Generators use triboelectric materials, and the Battery-free Game Boy and Peppermill which both harvested energy from user actions. Similarly, FaceBit can rely on the user to create energy (from breathing and movement).

There has also been prior work on health-related sensing. Sensing outside conventional medical settings to facilitate low-cost continuous health monitoring has long been investigated. For example, people have deployed sensors in environments to sense personal health. It is also possible to repurpose mobile devices for health sensing. One example is wearable bio-sensing. Wearable devices have the advantage of being close to a user's body. This proximity affords better signal quality, sensing coverage, and often improves comfort versus other devices. Recently, there has been commercial success for smartwatches and wristbands that feature bio-sensing, such as the PPG-enabled Apple Watch and Fitbit. There have also been brainwave sensors featured on headsets (e.g., Next Mind). In contrast with these sensing systems, the proposed sensing was confined to mask form factors, which makes the proposed work more suited for masks and PPE integration.

None of the previous works have investigated the intersection of energy harvesting, smart face masks, and on-device signal processing. Indeed, most methods demonstrate health metric capture by either relying on a phone and large battery, or processing data offline. However, these previous techniques in health based sensing are not as feasible for the realities of disposable, interchangeable face masks, which require a special form-factor and particularly high ease of use. No previous work has developed a smart face mask hardware platform, tuned to the unique constraints of on-mask sensing, or released the platform for use by the research community. As the COVID-19 pandemic continues, this is an unmet need.

A first goal of the system was to provide reliable and valid health sensing. Specifically, FaceBit was designed to provide reliable monitoring of the wearer's important health signals (e.g., respiration- and heart-rate), and should provide capability to expand to new health signals with expansion sensors and software. A second goal of the system was to enable real-time interventions. While accurate sensing is critical, having access to wireless data in real-time to make informed decisions or deliver a just-in-time adaptive intervention (JITAI) is a crucial feature for clinicians, workers, patients, and the general population. Therefore, the FaceBit platform is able to communicate with any mobile device, e.g., the wearer's smartphone. An informed decision was made to ensure that FaceBit is compatible with all types of mobile devices and provides a reliant and flexible interface.

Another goal for the proposed system is long battery lifetime and low bulk/weight. Charging and battery replacement are burdensome to the wearer. Long battery life enables near continuous monitoring of wearer's health markers and reduces abandonment. When a device can run for multiple days or weeks, new applications are enabled and interesting data and trends can be captured. However, large batteries make for large and uncomfortable sensors, and small size and weight is an important design criterion to allow for long term use. To accomplish this goal, techniques for energy-efficient runtime operation, and energy replenishment through energy harvesting were implemented in some embodiments. Another goal was for the system to be flexible, general purpose, research-ready, and easy to use. Face masks are ubiquitous passive wearables that, when retrofitted with FaceBit devices, could become important sensing platforms for future health studies in the medical and UbiComp communities. FaceBit was designed to be small enough to fit in the mask without bothering the wearer, to provide capability for general purpose sensing, to be easily reconfigurable in software and health metric collection, and to have associated tools and infrastructure that enable streamlined data collection and interpretation.

FaceBit is meant to be general purpose, however, healthcare professionals have the most risk, and the most constraints on PPE usage (via CDC and OSHA), so the inventors opted to inform the design of FaceBit prioritizing healthcare workers assuming that the general population would also benefit. To understand clinicians' needs for smart face masks and guide the platform design, the inventors conducted a study of 12 healthcare workers (4 medical doctors, 2 nurse practitioners, and 6 medical assistants) at a nearby pediatrics clinic. The inventors interviewed participants using a semi-structured interview that included a survey with a combination of 20 multiple-choice and free text questions.

First, the inventors asked a series of baseline questions to assess typical masking behavior before and during the COVID-19 pandemic. In response, 9 of the 12 (75%) healthcare workers reported never wearing a face mask before the pandemic. All 12 participants reported, while at work, always wearing a face mask (surgical) throughout the COVID-19 pandemic, and only removing the mask when eating. At the time of the study, N95 shortages were widespread, which may have played a part in the use of surgical masks.

The inventors asked each healthcare worker an open question about what features they would find helpful from a smart face mask. Common themes from these discussions included statistics about a mask's efficiency, such as airflow or when it is time to dispose of the mask, detection of exposure to COVID-19 or other viral infections, and vital health metrics such as heart rate and respiratory rate. Next, the inventors presented each clinician with a compiled list of 18 potential health metrics drawn from health guidelines and that are attainable by wearable devices based on prior literature. Each metric is considered a candidate for FaceBit based on the platform's current or future envisioned capabilities. These metrics can be categorized into three interest groups: mask information and safety (i.e, is the mask fitted and working), personal health (i.e., respiratory system signals and real time physiological monitoring), and environmental factors (i.e, air quality).

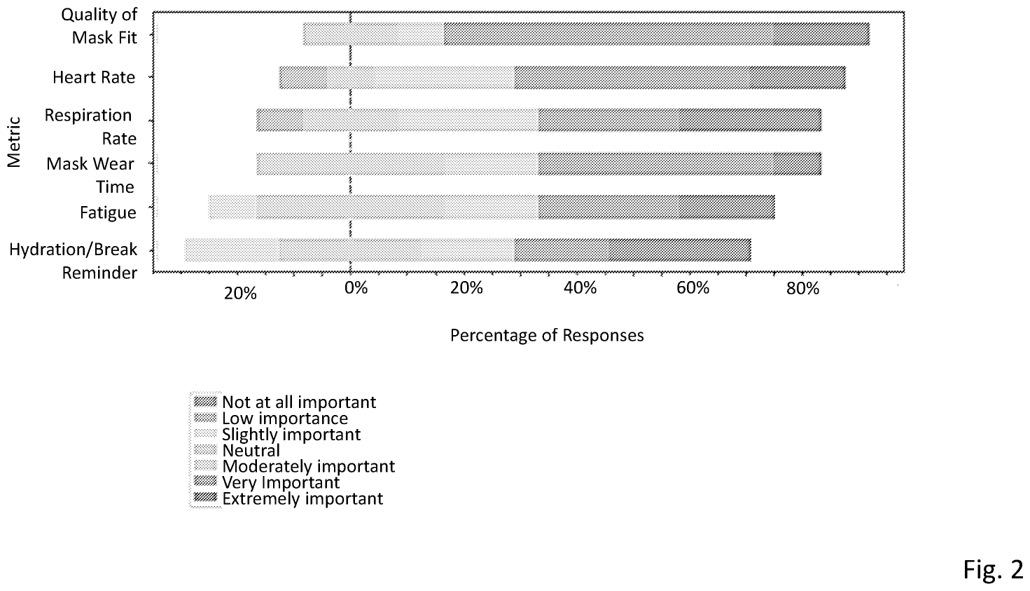

Clinicians ranked each metric on a Likert response scale. All metrics were considered important in some regard by the clinicians surveyed. For this study, the inventors chose to focus on metrics that were both of interest to clinicians and attainable by the first-generation implementation of FaceBit considering size, energy, and computational constraints. FIG. 2 is a diagram that shows survey results regarding the relative importance of various system metrics in accordance with an illustrative embodiment.

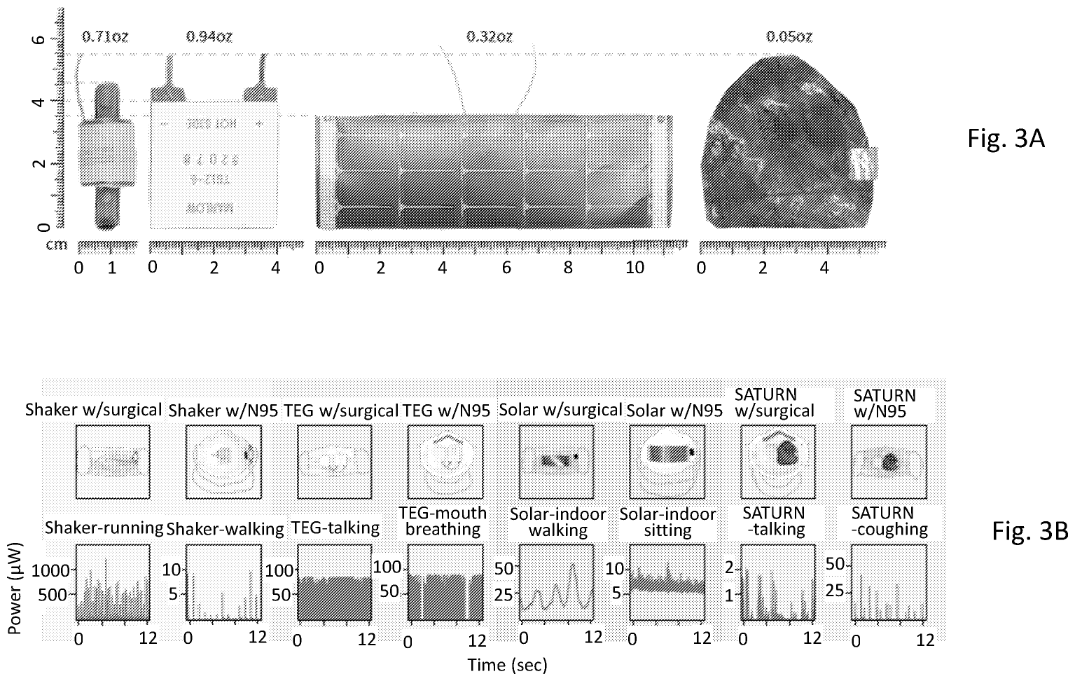

The inventors also investigated energy harvesting opportunities around the face to understand the potential for extending the battery lifetime of Facebit. Gadgets on the face are often exposed to ambient light, motion energy from head movements, and air flow which causes thermal and kinetic changes that can be harvested. The inventors considered routine activities that mask wearers perform in daily settings, and mapped these activities to harvesters—some were found off-the-shelf and others built from scratch. The design exploration, tradeoffs of each harvester, and the energy harvesting potential of daily activities are discussed below. For all experiments the inventors used a Rocketlogger device to record the energy harvested while an experimenter performed these activities with the mask on. The results of the exploration are shown in FIG. 3.

Specifically, FIG. 3A depicts various energy harvesters (shaker, thermoelectric generator (TEG), flexible solar panel, and SATURN) and their dimensions in accordance with an illustrative embodiment. FIG. 3B depicts power harvested from activities with different harvesters along with mask placement in accordance with an illustrative embodiment. To test harvesting energy from light, a paper-thick flexible PowerFilm MP3-37 4.49 [symbol] 11.44 in 150 mW flexible solar panel was attached to the outside of a face mask. A disposable surgical mask is 7 [symbol] 13.875 in. The inventors clipped the solar panel to surgical and N95 masks using small magnets and performed different activities such as walking indoors/outdoors on cloudy and sunny days, sitting indoors without a light source nearby, and sitting indoors near a light source. In alternative embodiments, a different type of solar film/panel may be used.

The inventors also tested energy harvesting via head movement. To the best of the inventors' knowledge, there exists no such harvester that can capture head movement and is small enough to be attached to a surgical mask. The inventors fabricated a lightweight harvester in the form of a shaker that generates energy from head movements. The shaker follows the principle of electromagnetic induction. In one implementation, a coil is made by winding a 42-gauge coated copper wire (typically used in motors and generators) on a small hollow plastic cylinder. A cylindrical magnet is placed inside the core with both ends blocked by caps. Another magnet, smaller than the one in the core, is attached to one of the caps, such that the core magnet and side magnet repel one another. When the harvester is shaken by head movement, the core magnet moves inside the coil. This movement changes the magnetic field around the coil, resulting in voltage induction (as per Faraday's Law of Induction).

Thermal energy was also explored. Face masks trap the heat expelled by breathing. A thermoelectric generator (TEG) can convert this heat to useful electrical energy. The inventors attached a 40.13-140.13-13.91 mm TEG TG12-6-01S to the inside of a mask with the help of magnets, as shown in FIG. 3B. As the wearer breathes, the side of the TEG facing the user becomes hotter than the other side, and this generates a very low voltage that can be boosted by a specialized DC/DC converter to a usable level. The inventors recorded how much energy can be harvested from activities like nasal breathing, mouth breathing, and talking.

Kinetic energy from breath was also examined for harvesting. The inventors used a triboelectric nanogenerator (TENG) to convert the kinetic force of breath into electricity. The inventors designed a variant of the SATURN platform, a TENG that is flexible and is made from conformable light-weight materials such as a film of dielectric (Fluorinated Ethylene Propylene), and a paper with tiny laser cut holes. Both the paper and the dielectric have a thin layer of copper deposited onto them, and are arranged on top of each other such that the dielectric is sandwiched between the two copper layers. Simple fabrication and a configurable shape (FIG. 3A) allows it to be embedded easily into different types of masks using simple magnetic attachments. FIG. 3B shows the TENG device embedded into both N95 and surgical masks.

As discussed above, FIG. 3A shows the weight and dimensions of all the harvesters used for harvesting energy from face masks. Overall, different energy harvesters have different sets of pros and cons, and a user may choose to simply not integrate an energy harvester. As expected, solar panels provide the highest output, even indoors, but might be considered aesthetically displeasing when placed on a mask. Thermoelectric harvesters provide a short burst of energy when first put on the face, but once temperature on both sides of the TEG equalizes, the energy generation lessens. TENG harvesters are the lightest, but also generate the smallest amount of energy among all options. Despite this low power output, the light weight and flexible form factor still make TENG harvesters an attractive option for incorporation into the mask. The Shaker outputs significant amounts of energy when its wearer is running (the second best harvester), however, it is heavier and bulkier than the other harvesters. The shaker harvests more when attached to a surgical mask. This is hypothesized because surgical masks are not as tightly fitted as N95 masks which amplifies the motion of the magnet.

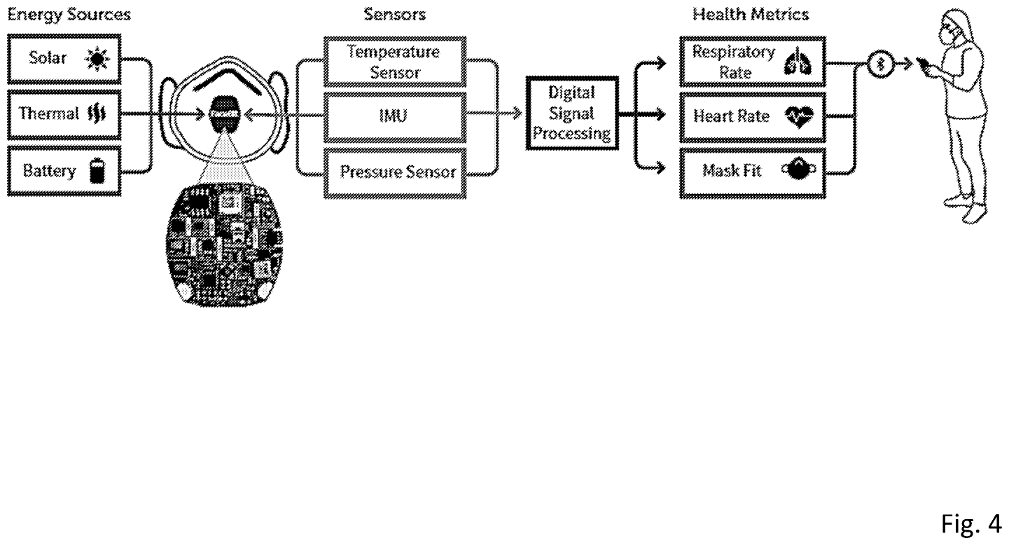

The hardware platform for FaceBit and its design are discussed below. FIG. 4 is a block diagram overview of the FaceBit hardware in accordance with an illustrative embodiment. As shown, data from multiple sensors is gathered and then processed on-device for health metrics. This is reported to the phone application as well. The circuitry is designed to automatically switch between a primary cell (battery) and harvested energy in the embodiment shown. In an illustrative embodiment, FaceBit includes a small printed circuit board (PCB) with microcontroller and sensor suite that attaches to any surgical, N95, or cloth face mask via a magnetic clip.

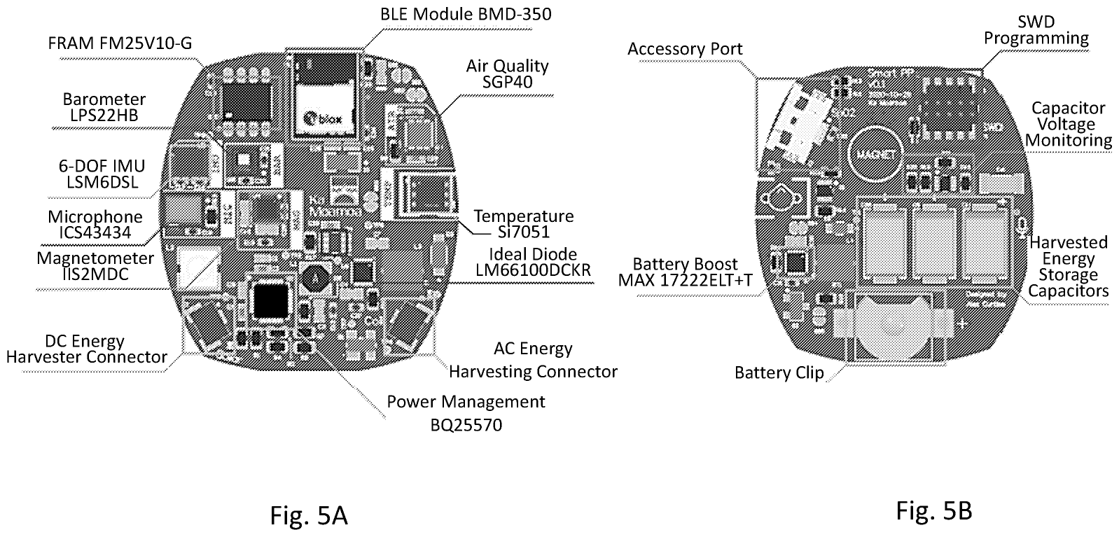

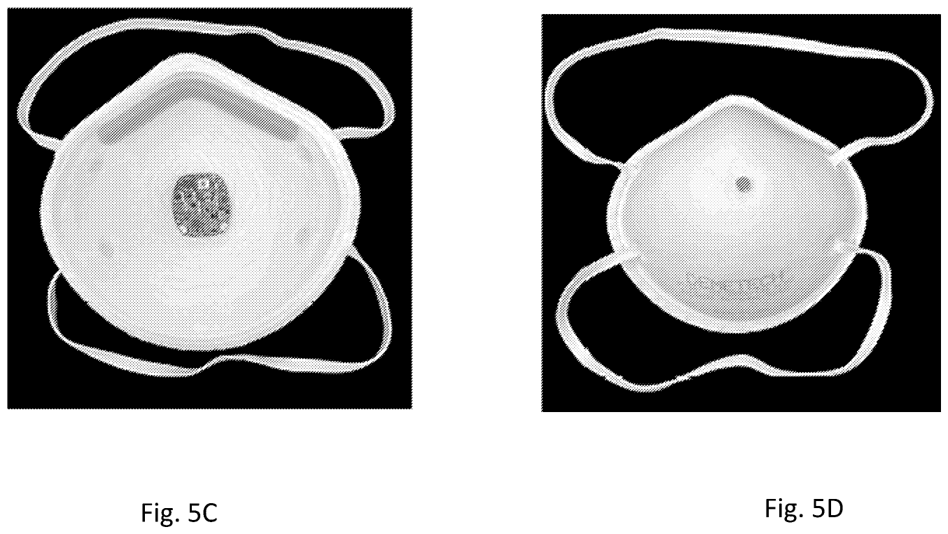

FIG. 5A depicts a top view of a circuit board for the proposed system in accordance with an illustrative embodiment. FIG. 5B depicts a bottom view of the circuit board for the proposed system in accordance with an illustrative embodiment. FIG. 5C depicts an internal view of the circuit board (FaceBit) mounted to a mask in accordance with an illustrative embodiment. FIG. 5D depicts an external view of the mask with a magnet used to secure the circuit board (FaceBit) in accordance with an illustrative embodiment. As shown, the top of the FaceBit board contains the computing components (i.e., processor, memory, transducer, etc.), sensing components, and most of the energy harvesting circuitry. In one embodiment, the application runs on the BMD-350 Module, which incorporates Nordic's NRF52832 (512 k Flash/64 k RAM). The bottom of the FaceBit board contains the energy storage elements and the programming interface. It also includes an accessory port which allows additional modules to be added in the future.

In alignment with the design goals, FaceBit is small enough to fit comfortably in either an N95 or surgical mask (1.2″ wide×1.3″ tall). The board features an NRF2832 SoC from Nordic Semiconductor, which is packaged with a chip antenna on the pre-certified BMD-350 BLE Module. With a goal of using this platform to explore a wide range of health and environmental sensing applications, this version includes six sensors. Only three are used herein: the LPS22HB barometer, the LSM6DSL 6-DOF Inertial Measurement Unit (IMU), and the Si7051 temperature sensor. In future versions, the number of sensors can be reduced, thereby reducing the size and cost of the board.

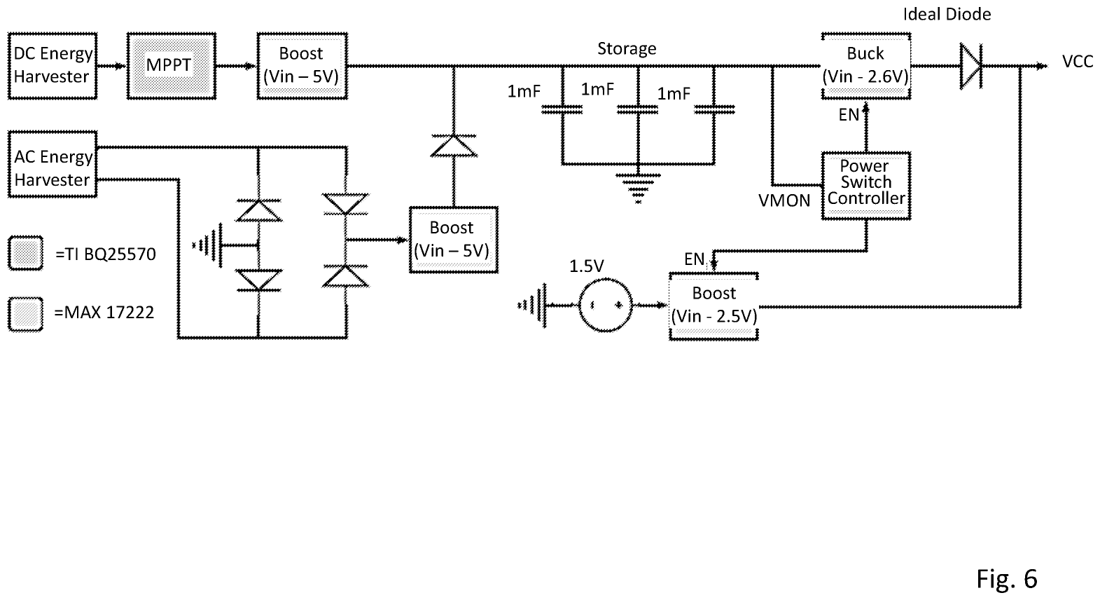

The inventors adopted a flexible hybrid approach with regards to power. FIG. 6 depicts a hybrid power architecture of the FaceBit platform in accordance with an illustrative embodiment. The circuit is able to power itself from either a primary cell or energy harvested from AC and DC sources. The system switches intelligently between these sources, favoring harvested energy when a sufficient quantity has been stored. A battery holder was included for a small, 105 mWh primary cell, but the system also includes the circuitry and storage to power the board using energy harvested from both DC (e.g. solar) or AC (e.g. shaker) sources.

Three tantalum capacitors on the bottom of the board combine to provide 3 milliFarads (mF) of storage capacity for harvested energy. These are charged by Texas Instruments' BQ25570 power management IC, which incorporates a boost converter with MPPT, intelligent charging circuitry, and a buck converter to reduce the voltage from the storage elements to a level tolerated by the system. The tantalum capacitors are charged to 5V (80% of their 6.3V rating), and so are able to store a total of 10.4 μWh. The inventors use the “power good” indicator on the BQ25570 IC to implement an intelligent switch that automatically decides which source to draw power from based on the voltage of the capacitors. By default, the system draws its power from the battery (when one is present). When the storage capacitor voltage exceeds a resistor-programmed threshold (3.0V in the application), the BQ25570 begins to draw from them and the battery boost converter is disabled, which protects the battery from reverse current.

When the storage capacitors fall below 2.6V, the BQ25570 switches off its buck converter and system power is once again drawn from the battery. An ideal diode (LM66100DCKR) prevents reverse flow into the energy harvesting circuitry while the battery boost converter is active. This power architecture allows for a relatively long battery life (the energy density of primary cells being greater than that of rechargeable cells), while still maintaining the ability to store and use harvested energy. The device can also operate in a mode where it uses only harvested energy, activated simply by not placing a battery in the connector. This flexible architecture suited the development process, but it is also possible to further simplify and miniaturize the board by using either only a rechargeable cell or only energy storage capacitors.

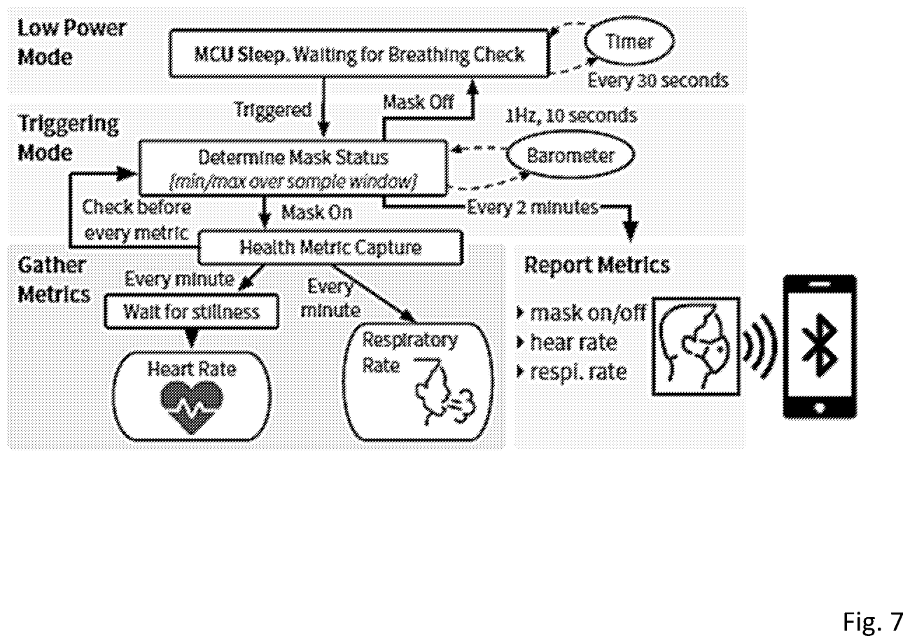

Unlike other systems, the health metrics can be computed in real time on the device itself. This requires careful attention to energy cost so as to conserve battery life. This starts with the energy-efficient runtime which eliminates the need to manually turn FaceBit off or on each time a mask is donned or doffed. FIG. 7 is an overview of the energy-efficient runtime of the smart mask system in accordance with an illustrative embodiment. The runtime system saves energy by using body signals to guide when to turn on sensors, process data and metrics, and report those to the phone. All operation frequencies are configurable. Once it is established that the mask is being worn, the remaining tasks (heart and respiratory rate sensing, BLE broadcast) are scheduled on a timer. These intervals can be adjusted to fit the application's needs. FIG. 7 shows the intervals used in the evaluation, although different intervals can be used in alternative embodiments.

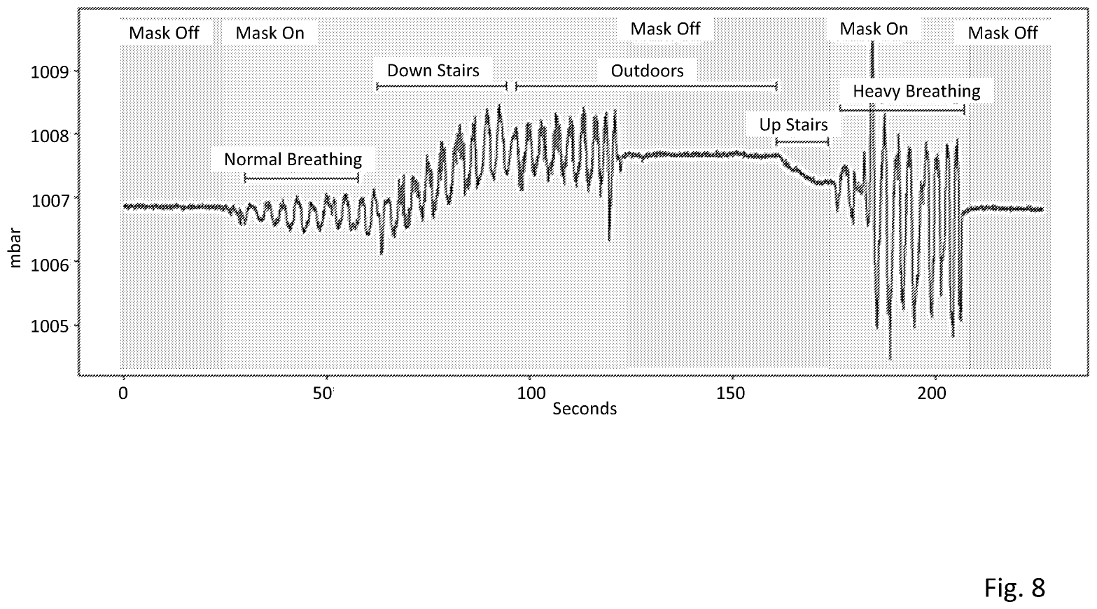

With respect to sensing, the inventors focused on sensing health metrics that are technically feasible for commodity sensors, that have a definitive ground truth to compare against, and that are of interest to clinicians as identified by the study. These health metrics are wear time, heart rate, respiration rate, and mask fit. In order to compute wear time one must first determine if the mask is on or off. This can be accomplished using the difference in the pressure signal over a short time window that contains at least one respiratory cycle. A key observation is that the standard deviation of a pressure signal when the mask is on is much higher than when the mask is off, due to the pressure differences caused by respiration. This can be further simplified by only retaining the minimum and maximum pressure values within a time window. This difference, in combination with a minimum threshold, allows one to clearly see state changes in the mask. FIG. 8 depicts an example pressure signal which follows a user through a normal sequence of activities in accordance with an illustrative embodiment.

In idle mode, the device wakes up from sleep intermittently to check if it is being worn. Since this task is performed often, it is important that it is low power. With this in mind, the board uses only a barometer and reads data from it slowly at 1 Hz, sleeping between measurements. It records ten samples over ten seconds, keeping track of the minimum and maximum values. If the difference between these two is greater than 0.1 mbar, it assumes the mask is being worn. Since spurious spikes in pressure can occur when the mask is off, this check is performed before every health measurement task. These data-points can be aggregated to calculate a running wear time, but in order to determine wear time of any particular face mask, user interaction is required to track disposal or switching. Therefore, wear time is calculated by both the sensor board and via user interaction with the mobile application.

Heart rate is an important physiological parameter. An elevated resting heart rate is associated with increased incidence of cardiovascular disease in both men and women. Heart rate also rated highly in the healthcare worker survey. Unfortunately, traditional methods of heart rate detection (e.g. ECG, photoplethysmography) require constant, immobile contact with the skin which is unlikely in the context of an easy to don and doff mask. Remote imaging methods such as remote PPG were considered, however it was believed the unstable lighting conditions encountered in everyday life would confound those efforts, and additionally the need for a bright light source or otherwise a sensitive photodetector or camera would raise the cost or energy budget of the platform. As such, the inventors chose to explore another non-contact method that would allow for the gathering of heart rate data at various points throughout the day.

Ballistocardiography (BCG) is a method of cardiac health assessment whose origins date back to the turn of the 20th century, and was significantly refined by Starr, et al. in 1939. The historical methods relied on complicated mechanical setups involving swinging, free-hanging tables or chairs suspended from heavy springs, but they took advantage of the same basic phenomenon relied upon in the FaceBit heart rate detection algorithm: with each heartbeat, blood is ejected forcefully out of the left ventricle of the heart into the major arteries of the body. This causes a subtle “recoil” throughout the body, which can be detected either by using mechanical contraptions, or by sensitive accelerometers like the LSM6DSL found on the FaceBit circuit board. Each ballistocardiographic pulse is composed of several high-frequency waves, which are hypothesized to arise from interactions between regions of higher and lower pressures in the ascending and descending aorta during each heartbeat. The intervals and amplitudes of these individual waves in the BCG complex are thought to correlate with various cardiovascular health markers. The pre-processing approach adopted by FaceBit exploits this characteristic structure to better locate each heartbeat in the raw 3-axis gyroscopic data, which is fundamentally a noisy signal in a wearable context.

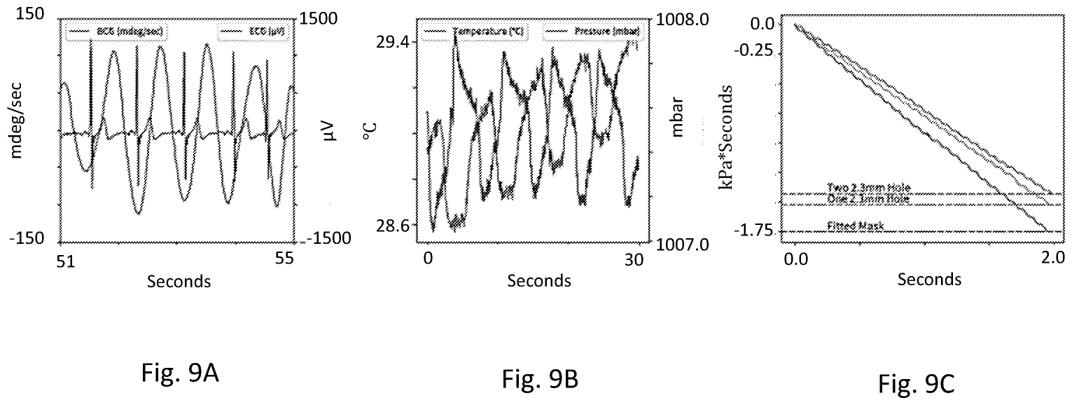

FIG. 9A shows a recovered BCG/heartbeat signal obtained from a seated participant wearing an N95 mask fitted with the FaceBit, along with an ECG signal recorded at the same time in accordance with an illustrative embodiment. Each peak in the BCG signal represents a collection of tiny micro-motions of the head caused by the heartbeat represented by the peak in the ECG signal directly preceding it. In FIG. 9A, the ECG ground truth signal is collected from a Polar H10 chest strap compared to a Ballistocardiography (BCG) signal extracted from the FaceBit's gyroscope.

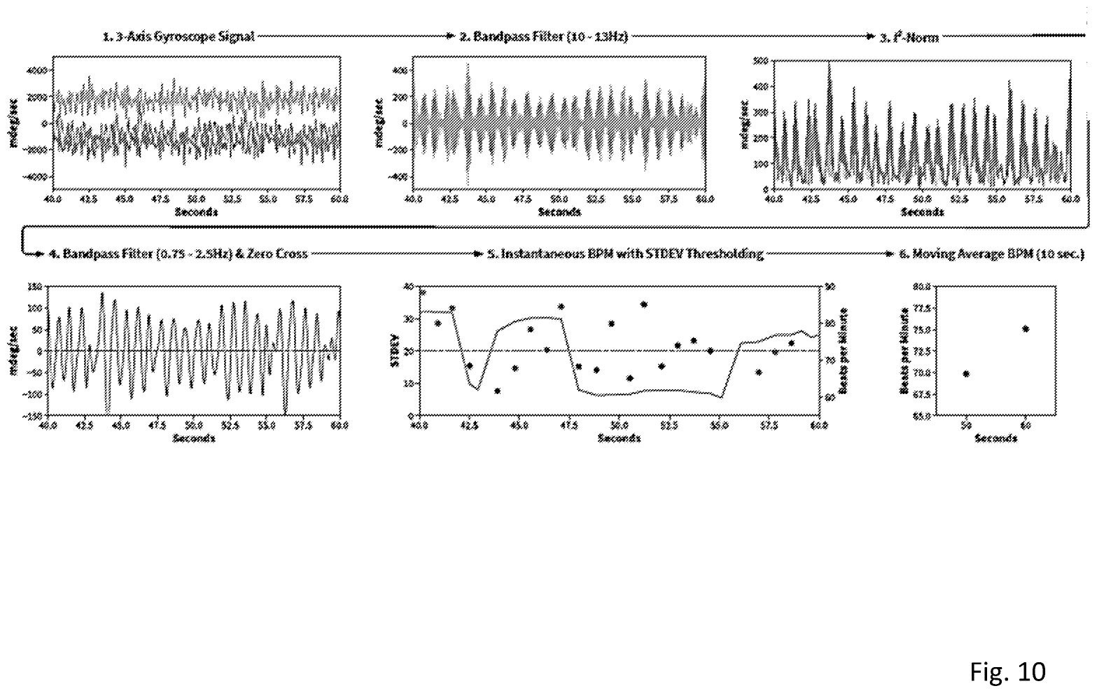

FIG. 10 shows a signal processing pipeline for the proposed system in accordance with an illustrative embodiment. The algorithm was implemented entirely on the NRF52832 in C++ to run in real-time. First, 3-axis gyroscope data was collected at 52 Hz. The inventors then applied a fourth-order IIR band-pass filter with cutoff frequencies of 10 and 13 Hz. This passband removes the DC component and low-frequency motion artifacts (e.g. respiration), but retains the high frequency pressure waves associated with the BCG waveform complex and the time between each zero cross of the signal such that instantaneous heart rate is extracted. Namely, the I, J, K, and L waves of the typical BCG waveform occur in this frequency range. The signals from each of the three axes are then combined via the euclidean norm (or 1 2-norm). Notably, this operation allows the algorithm to function with the FaceBit board placed in any orientation relative to the wearer. Next, the 1 2-norm signal is fed through a smoothing, second-order IIR band-pass filter with cutoff frequencies of 0.75 and 2.5 Hz, corresponding to a range of 45-150 beats per minute. This is the recovered BCG-HR signal. Additionally, a standard deviation threshold is used to determine realistic change in rate from beat-to-beat. In one embodiment, heart rate is calculated as a 10-second average of valid instantaneous rates.

The FaceBeat algorithm (or smart mask application) is tasked with extracting heart rates in real time from this signal, which is easily corrupted by motion artifacts from everyday living like walking or even just looking around. Moreover, the heavy filtering involved in the pre-processing stage tends to produce signals which resemble valid heart rates when none are present. The FaceBeat algorithm attempts to exclude regions of data that it suspects are due to motion and phantom signals, and use only the regions that correspond with the wearer's true heart rate. In this way, FaceBit is able to detect the user's heart rate during moments of stillness throughout the day.

To accomplish this, the algorithm first detects and stores the timestamps of descending zero-crosses of the detected signal. When five such timestamps have accrued, the instantaneous heart rate associated with these timestamps is calculated by 1) measuring the time difference between consecutive elements, yielding [seconds]/[beat]2) taking the reciprocal of this number to obtain [beats]/[second] and 3) multiplying the reciprocal by 60, thereby arriving at [beats]/[minute]. At this point there are four instantaneous heart rates associated with the last five suspected heart beats. The inventors rely on the innate predictability of the heart and the inter-beat interval to conclude whether these instantaneous heart rates are genuine or the product of motion artifacts. Specifically, the standard deviation of the four instantaneous heart rates was calculated, and if they fall below a threshold (e.g., 20 beats/min were used in the initial evaluation), their average is recorded as a valid heart rate. To save energy in the application, the FaceBeat algorithm is set to run intermittently. Initially, all valid heart rates (>45 BPM and <150 BPM) recorded within a given window were averaged together, and it is this average that was reported to the mobile application.

After an initial in-lab evaluation, the FaceBeat algorithm was refined. The standard deviation cut-off (threshold) was lowered from 20 beats/min to 7 beats/min. The inventors also used the amplitude of the BCG waveform as a discriminating factor. Specifically, it was noted that the amplitude of valid BCG waveforms are typically quite small, less than a maximum of 100 millidegrees/second. This is easily dwarfed by normal body motion, vehicular vibrations, and other everyday phenomena. Therefore, the inventors added a fourth step to filter out suspected beats whose amplitudes exceed a given threshold (150 millidegrees/second in the scenario-based evaluation). Finally, the inventors began reporting any valid instantaneous heart rates rather than an average in order to better compare results with the ground truth.

It is noted that the rejection of all motion artifacts is complex, particularly those that fall within the 10-13 Hz window used by the first band-pass filter and especially the subset of those which are periodic in the same range as heart beats. In response, the inventors designed the motion-aware approach to only report user heart rate when the motion artifacts are sufficiently small. Additionally, physiological differences (height, weight, cardiac stroke volume, cardiac arrhythmias) between users and within individual users over time may require adjustments to the thresholds used in the FaceBeat algorithm. A promising remedy is personalization of the algorithm to each user via an initial calibration routine carried out in conjunction with the FaceBit mobile application. Also, the location of the FaceBit circuit board within the mask or differences between masks (e.g. stiffness, size) may alter the raw gyroscopic signals the algorithm uses to detect heartbeats. Taking the euclidean norm of these filtered signals is thought to make the method robust to these effects.

Another metric considered is respiratory rate based on in-mask temperature changes. In an N95 (or equivalent) type mask, normal breathing results in a pressure drop over the filter material that the FaceBit's pressure sensor can easily detect. However, loose-fitting surgical or cloth masks do not generate as large a signal, especially when placement of the FaceBit board is up to the user. Fortunately, breathing also causes the temperature in a mask to fluctuate with a distinct periodicity, and this effect is highly conserved between mask types. Moreover, the thermal mass of the board and surface-mount temperature sensor acts as a sort of low-pass filter, and the insulation properties of still air serves a rudimentary peak-hold function which amplifies the effect of moving air (and therefore breaths). For all these reasons, the on-board temperature sensor can be used as the source for respiratory rate detection.

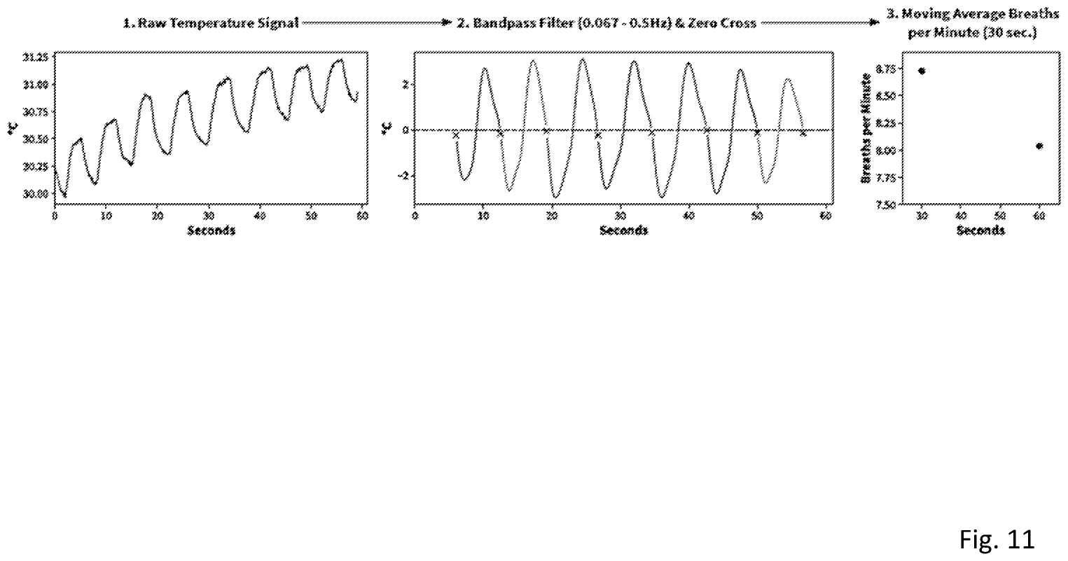

FIG. 9B shows the signal obtained from FaceBit's temperature sensor and pressure during tidal breathing in an N95 Mask in accordance with an illustrative embodiment. The temperature signal is shifted relative to the pressure signal due to the thermal mass of the circuit board and temperature sensor, but the frequency is identical. The respiratory rate algorithm begins by collecting temperature readings from the Si7051 temperature sensor at 10 Hz. The raw values are sent through a second-order IIR band-pass filter with cutoff frequencies at 0.067 and 0.5 Hz, corresponding to limits of 4 and 30 breaths per minute (BrPM). This filters out the DC component (ambient temperature) as well as trends that might result from donning the mask or entering a room with a different temperature. Next, the timestamps of any descending zero-crosses are buffered. At the end of the sampling window (e.g. 30 seconds), the respiratory rate is calculated from the average time difference between zero-crosses (i.e. length of each breath). Any breaths that correspond to a respiratory rate faster than 30 BrPM or slower than 4 BrPM are excluded from the final calculation, as these are likely to have occurred during speech or other disordered breathing. FIG. 11 shows an example raw signal and the output of the respiratory monitoring algorithm in accordance with an illustrative embodiment.

The respiratory monitoring algorithm was updated after an initial evaluation to achieve better performance. To measure high respiratory rates that may occur during exertion, the inventors expanded the filter passband to allow frequencies from 0.067 to 1 Hz (4 to 60 BrPM). The inventors also included a temperature threshold that the signal must exceed to register as a breath (0.04° C.) in order to reduce the classification of small oscillations that arise from the filter as valid breaths.

There are several variables which can potentially affect the performance of the respiratory monitoring algorithm. Sensing respiration rate when the user is talking can affect the output of the algorithm. To account for this, a review of potential advanced filtering methods is included below. Also, poorly constructed masks often yield a lower signal-to-noise ratio, as the temperature difference between the inside and the outside of these masks is much lower than that seen in high-quality masks. In practice, it was found that positioning the FaceBit near the center of the mask mitigates this problem since it puts it directly in the path of each breath. Temperature variation within N95 and surgical masks is discussed below.

It is also noted that as ambient temperatures approach the user's exhaled breath temperature, signal quality is expected to decrease. Exhaled breath temperature, of course, depends on ambient temperature but reaches a maximum of approximately 32.5° C. As such, respiratory rate may not be reliably measured by this method in warm environments. In these conditions, the pressure sensor may provide a cleaner signal, suggesting a sensing algorithm that leverages both signals in the future. Since the signal processing pipeline functions equally well for pressure data, it would be straightforward to have a sensing pipeline that switches its signal source between pressure and temperature.

Mask fit was the most important metric to the clinicians surveyed as part of the above-discussed study. This is understandable because the difference between a well- and a poorly-fit mask can have significant health-related implications. However, mask fit is a difficult parameter to measure, and currently requires bulky, awkward, and expensive test equipment. Initial results from the platform showed a promising correlation between leakage and the integral of the pressure signal while a mask was under suction from an attached vacuum pump. A well-fit mask presents a higher resistance to airflow, and so the slope of this integral can serve as a straightforward indicator of mask fit. FIG. 9C depicts cumulative integration of the pressure signal sensed inside an N95 mask while under suction from a 12V vacuum pump in accordance with an illustrative embodiment. Minimum values highlighted represent a measure of mask resistance to airflow.

However, in practice this could be a cumbersome method to detect mask fit. The vacuum pump is large, loud, heavy, and requires a port that passes through the mask. Instead, in keeping with the aim to achieve a low user burden the inventors avoided any mask modification or additional hardware, and rely solely on the user's lungs to pass a constant volume of air through the mask using a special breathing exercise which could be facilitated by the mobile application. A mask fit score is calculated using a signal processing pipeline in conjunction with this exercise. First, the user is asked to exhale as much air as they can from their lungs (reaching their “residual volume”), and then hold their breath for five seconds. This breath-hold allows the inside of the mask to equilibrate with ambient pressure, which is recorded and used as a baseline. The user is then asked to inhale as deeply as possible at a normal rate until their lungs are filled as much as possible, and then hold their breath for another 5 seconds to obtain another atmospheric pressure reading. This exercise (breathing in as much air as possible after breathing out as much air as possible) is also known as an inspiratory vital capacity test. Vital capacity is a static lung volume, and changes only slowly over time with age (though it can be affected in the short term by posture or disease conditions).

Pressure inside the mask is sampled at 50 Hz throughout the fit test. Atmospheric pressure (obtained at the beginning and end) is subtracted from the pressure readings to obtain gauge pressure. The area under this curve [Pa*s] is proportional to the total volume of air inhaled, with mask resistance [kg⁻¹ meter ⁴second] as the proportionality constant. Because the total volume of air inhaled is unknown, and is necessary to compute a value for mask resistance, the inventors asked the user to calibrate the device by pressing the mask against their face to ensure a good seal. It is noted that this calibration needs only to be performed once per mask type per user. Future mask fit scores are calculated as a percentage of this initial calibration test.

The size of the leak and the consistency of inspiratory vital capacity are the two variables that affect this algorithm. An initial consideration is what size leak needs to be detected to be useful. It has been found that a mask with two leaks (3 mm diameter each) allowed a total inward leakage of 10.67%±4.6% (ratio of particle concentration inside the mask to outside the mask). This, compared to 0.31%±0.4% for a respirator with no leaks. Four leaks (3 mm diameter) increased the TIL to 30.54%±4.2%. Meanwhile, OSHA requires a fit factor greater than 100 (less than 1% TIL) during quantitative fit testing procedures, and so one can piece together that they should strive to detect leaks smaller than 3 mm (×2). It isn't clear from the available literature, however, what the absolute minimum allowable leak size is. Inspiratory vital capacity can change with posture, however this can be mitigated by guiding the user through the exercise.

The FaceBit companion application is a phone and desktop application that serves two purposes. First, it is the research tool that allows interaction with the FaceBit sensor board. All evaluations utilize the FaceBit application for data collection. Second, the application serves as a proof-of-concept user interface for a Smart PPE platform.

The FaceBit companion application is developed for iOS and macOS using the Swift Programming Language. The user interface is written with the SwiftUI framework and utilizes Mac Catalyst, which supports iOS applications deployed on macOS. The app communicates with the FaceBit board via Bluetooth Low Energy (BLE) using a custom GATT profile. The application handles both high frequency time-series data stream for debugging, as well as low frequency computed metrics from the sensor board for actual deployment. Data is stored in a local SQLite database in one embodiment.

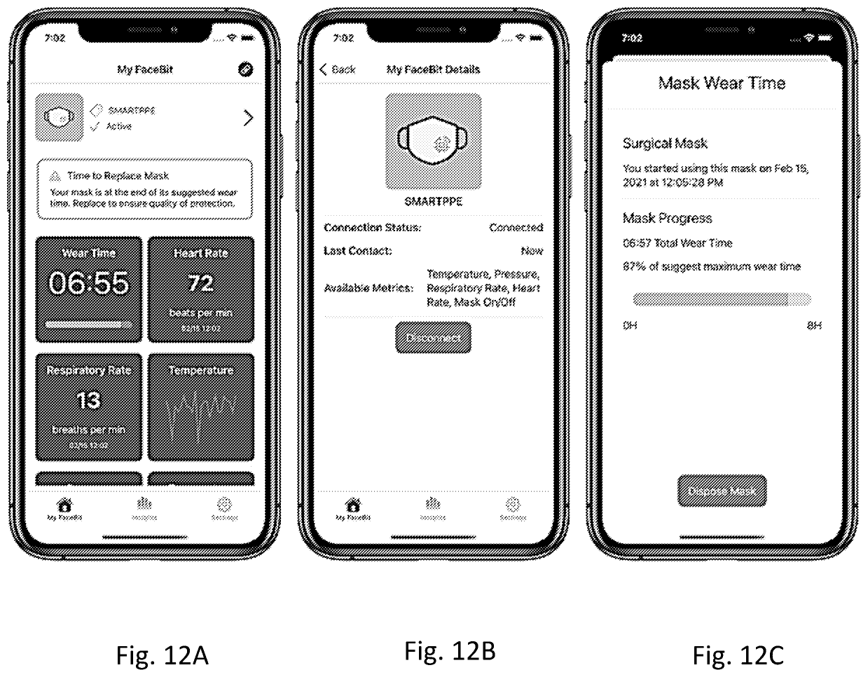

The inventors designed three sections of the app to demonstrate consumer interaction with FaceBit and two additional sections to assist in research. The user interface is compartmentalized such that user interaction does not interfere with data collection or vice-versa. FIG. 12 depicts the application's home screen, sensor device details, and mask wear-time interface that allows for tracking of the replacement of a mask via user logging and wear time detection. Specifically, FIG. 12A depicts that FaceBit phone application homepage displaying general details, current wear time, respiration rate, heart-rate, and temperature data from the FaceBit sensor board in accordance with an illustrative embodiment. Also shown is a wear time warning indication suggesting a mask change. FIG. 12B depicts the mask wear time interface for tracking mask disposal in accordance with an illustrative embodiment. FIG. 12C is the FaceBit sensor board detail screen outlining current connection status in accordance with an illustrative embodiment.

The homepage is a dashboard for at-a-glance information about the state of FaceBit. Notifications can be displayed or pushed to the user, and goals set for the desired amount of wear time. Additionally, this time-tracking interface can assist other reminders such as hydration or break reminders, and notifying the user the mask has been worn for various continuous lengths of time. The vision is that a user will be able to customize the home screen with desired widgets to receive timely information on the signals of interest. Additionally, developers will be able to easily add new widgets. Based on feedback from healthcare workers, the inventors developed widgets for respiratory rate, heart rate, and wear time. The inventors also included widgets for raw temperature and pressure readings. In alternative embodiments, different sensors and/or functionality may be incorporated into the system.

The FaceBit platform also provides capabilities that support research in the ubiquitous and mobile computing research community as well as basic clinical health research. To support the evaluation and exploration of the FaceBit platform, the research team developed a set of tools and a data-pipeline methodology to streamline research, including an event recording interface for the application, a data explorer on the phone application, and a streamlined evaluation system integrated with gitlab. These tools, documentation, source code, and hardware files are hosted at facebit.health.

The inventors also conducted an extensive evaluation covering many aspects of the FaceBit platform. The goal of the evaluation was to measure the capabilities of FaceBit, test the robustness and performance in the face of a diversity of situations, identify the places where improvement is needed, and understand the impacts of various factors on performance, battery lifetime, and user burden.

An experimental campaign was conducted to explore the following considerations in the evaluation. A first consideration is the accuracy of key FaceBit metrics in varied and confounding in-lab scenarios, such as respiratory rate, heart rate, and mask fit. Another consideration is how the FaceBit performs in noisy free-living scenarios such as riding in the car, on a train, or walking down a busy street. Another consideration is the impact of device location and mask type on bio-physiological signal quality. Another consideration is the impact of human/physiological differences on signal quality. Another consideration is the energy consumption and battery lifetime of FaceBit. Yet another consideration are the barriers and facilitators for user burden and comfort.

The inventors conducted an in-lab study to understand the accuracy of the heart and respiratory rate metrics, calculated on-device. The inventors then evaluated calculating mask fit based on leak detection in a mask. The evaluations of respiratory rate and heart rate were completed in unison, with FaceBit wirelessly transmitting both metrics to the FaceBit phone application during a set of activities performed in sequence by each participant. The evaluation included nine graduate and undergraduate participants (4 female), however, one participant's data was lost due to a ground-truth device error, therefore the report is based on eight participants.

All participants wore an N95 respirator (Demetech, Cup-Style) with an attached FaceBit device running the application firmware. Participants also wore a Polar heart rate monitor chest strap (H10), and a NeuLog respiration belt with a USB data logger (NUL-236, USB-200). FaceBit data, Polar heart rate data, and NeuLog data were collected using the FaceBit phone application (on macOS), a custom iOS application using Polar's BLE SDK, and NeuLog's experimentation application, respectively. Due to a documented low-power-mode related bug in the mbed BLE stack, FaceBit broadcast data to the companion application every 2-minutes and then reset.

Participants were evaluated for a total of 25 minutes, excluding setup and transition times. To obtain initial understanding of the reliability of the signal, the inventors designed a three-phase structured study involving sitting (ten minutes), activity (two rounds of one minute of squats followed by four minutes of sitting), and standing (five minutes). While seated and standing, participants watched a documentary playing on a laptop screen. Evaluation with six participants took place outside on a university campus and three inside a lab space. One participant recorded inside was excluded due to an error in the NeuLog respiratory sensor data capture (the signal recording stopped mid-session). The evaluation followed a strict protocol for consistency and sanitation purposes, including following COVID-19 guidelines.

The Neulog respiration belt was fit around a participant's mid-section and recorded an arbitrary analog signal corresponding to the pressure of the belt's air-bladder. Data from the respiration belt was excluded from evaluation during air-squats and transitions since the belt's air-bladder compressed when moving from a standing to sitting position, resulting in inaccurate estimates. FaceBit attempted to measure respiration rate once per minute with a 30-second window of data.

The respiration belt collected data at 5 Hz. Post-experimentation, ground truth respiration rate was calculated from this signal by first passing the signal through a Savitzky-Golay filter for smoothing. Peaks were then identified using an empirically set prominence value of 50 [Arb Units], a window length of 5-seconds, and a minimum peak-peak distance of 1-second. An instantaneous breaths-per-minute (BrPM) rate was calculated at each peak of the respiration belt signal, defined by the length of time in seconds since the previous peak. To match FaceBit's data sampling window, one can define the ground truth respiration rate as an average of instantaneous estimates over the previous 30-seconds at the time of a FaceBit measurement.

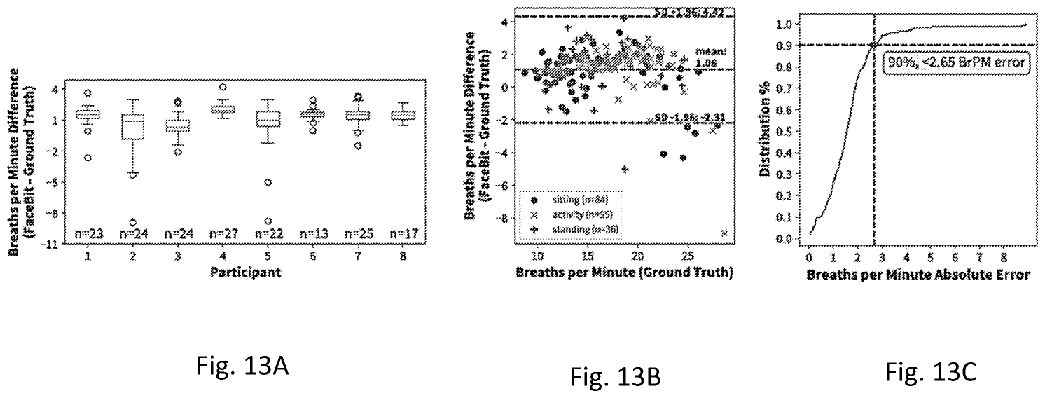

FIG. 13 depicts results for the 8 participants (186 samples). FIG. 13A is a box-plot of each participant in the respiratory rate evaluation, highlighting number of samples and outliers in accordance with an illustrative embodiment. FIG. 13B is a Bland-Altman plot comparing FaceBit's error to ground truth, and showing a tendency to report slightly above the ground truth and to be less accurate with higher ground truth reports in accordance with an illustrative embodiment. FIG. 13C shows the cumulative distribution function over the evaluation samples in accordance with an illustrative embodiment. On average, FaceBit reported a respiratory rate once every 55 seconds. The mean difference is 1.06 BrPM, demonstrating that on average FaceBit's algorithm reports slightly higher respiration rates than ground truth. FaceBit reported 90% of recorded samples within +/−2.65 BrPM of ground truth. It is noted that manual calculations of respiratory rate fall within 1 bpm error by definition, counting the number of chest raises over one minute.

After analyzing the results, the inventors identified explanations to account for the error seen compared to the ground truth. First, the algorithm for calculating ground truth is not perfect and does not account for anomalies that produce peaks in the signal that are not breaths, such as throat clearing, coughing, or talking. After exploring the outliers in detail, small peaks in the ground truth signal were discovered indicating some irregularity in breathing or perhaps a shift in weight on the air-bladder; this would cause the respiratory rate to increase briefly. Second, the test was limited to a 5 Hz signal frequency by the NeuLog software, which could cause shifted peaks, especially in higher frequency breathing. Additionally, after evaluation, the inventors discovered a firmware bug which caused some respiratory rate calculations to overflow on the sensor board. Most of these values were easily identified in post-processing, as they fell below a minimum valid respiratory rate (4 BrPM). The outliers in the −8 to −9 range it is suspected are due to this bug since it occurred when ground truth reported a high respiratory rate after a set of squats.

To test heart rate, a Polar H10 chest strap was fitted around each participant at the sternum and adjusted to the recommended tightness. Two streams of data were collected from the Polar device using a custom mobile application developed for this evaluation. The two streams include live heart rate (as calculated by Polar), and a live ECG trace. The Polar data streams were timestamped in order to sync them with the FaceBit data. Polar live heart rate was reported on average every 1 second, and ECG was recorded at a rate of 130.14 Hz. During the evaluation, FaceBit attempted to measure heart rate once every minute with a sampling window of ten seconds. FaceBit will only report heart rate if the algorithm is confident in the reading, otherwise it will broadcast a null result, indicated by a value of 1. FaceBit reported a null value for 14 of 163 total sampling periods, and 2 participants accounted for the majority of these null reports.

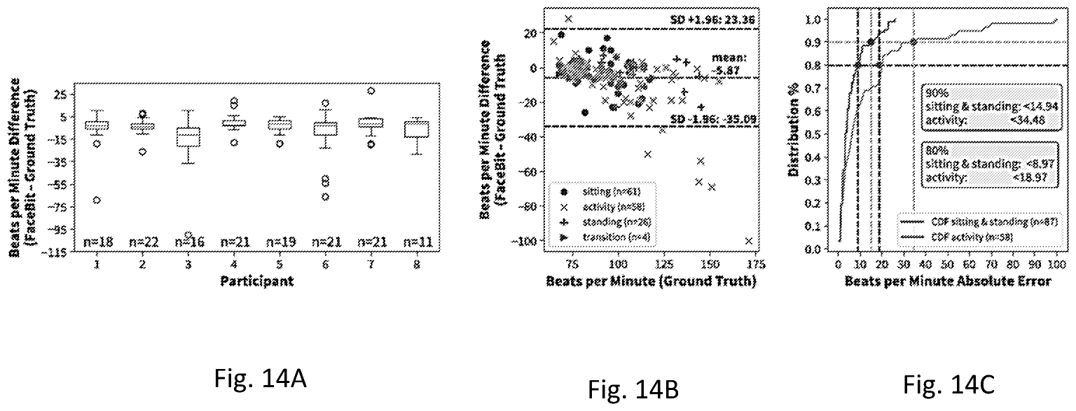

Every heart rate reported by FaceBit is an average of any valid heart rates recorded over the last ten seconds. Therefore, the inventors compared each FaceBit heart rate with an average of all recorded Polar-calculated heart rates over the same ten seconds. Results for the 8 participants are shown in FIG. 14. FIG. 14A is a box-plot of each participant in the heart rate evaluation, highlighting samples per participant and outliers in accordance with an illustrative embodiment. FIG. 14B is a Bland-Altman plot showing FaceBit reporting better accuracy in the lower heart rate ranges in accordance with an illustrative embodiment. FIG. 14C shows probability and cumulative distribution function over the evaluation samples in accordance with an illustrative embodiment.

The mean error over the course of the evaluation was −5.87 beats per minutes (BPM), and the standard deviation of the error was 14.91 BPM. In general, the heart rates reported from FaceBit during the sitting and standing test conditions were more accurate than during the activity condition, with 90% of reported heart rates being less than 14.95 BPM off from the ground truth rate, and 80% being less than 8.97 BPM off. The largest errors were seen during the activity condition (FIG. 14B), where 90% of the results fell within 34.48 BPM of ground truth, and 80% fell within 18.97 BPM.

Thus, a significant percentage of FaceBit reported heart rates are within 5 BPM of ground truth (68% of measurements in the seated and standing conditions, 43% of measurements in the activity condition). With that said, the evaluation pointed to a need for enhanced rejection of motion artifacts. There are a number of significant outliers in the sitting and standing conditions (20 BPM different from ground truth), and more significant outliers in the activity condition (>50 BPM). Lower accuracy at rates approaching and above 150 BPM are to be expected due to the second bandpass filter whose cutoff point lies in this range. There is also likely more motion from heavy and rapid breathing that often accompanies these faster heart rates.

An interesting finding is that the proposed algorithm, on average, underestimated the heart rate. Even after removing outliers greater than 1.96 standard deviations from the mean, the mean difference (FaceBit−ground truth HR) was −3.71 BPM. An observation from testing during the development of the algorithm was that it detected heartbeats more successfully during exhalation than inhalation. Moreover, respiration is known to affect hemodynamics in that inhalation decreases arterial pressure and increases heart rate through a series of physiological changes. This drop in arterial pressure during inhalation may reduce the amplitude of the BCG waveform enough to prevent detection in some cases, which in turn may bias the detection of heart beats during exhalation when arterial pressure increases and (importantly) heart rate slows.

To explore the feasibility of leak detection on the FaceBit platform, the inventors conducted two exploratory studies using raw pressure data from FaceBit and an external differential pressure sensor module. Both studies used an N95 mask fitted with three plastic bulkhead fittings with an internal diameter of 2.3 mm (4.2 mm²), to which the inventors attached a short length of silicone tubing with a removable end-cap to simulate a small mask leak. During each experiment, participants performed a self-guided inspiratory vital capacity test (IVCT) where they exhale their lung capacity, hold their breath for 5-seconds, inhale their total lung capacity, then again hold their breath for 5-seconds. It is hypothesized that one can use this relatively constant volume of air to quantify mask leakage.

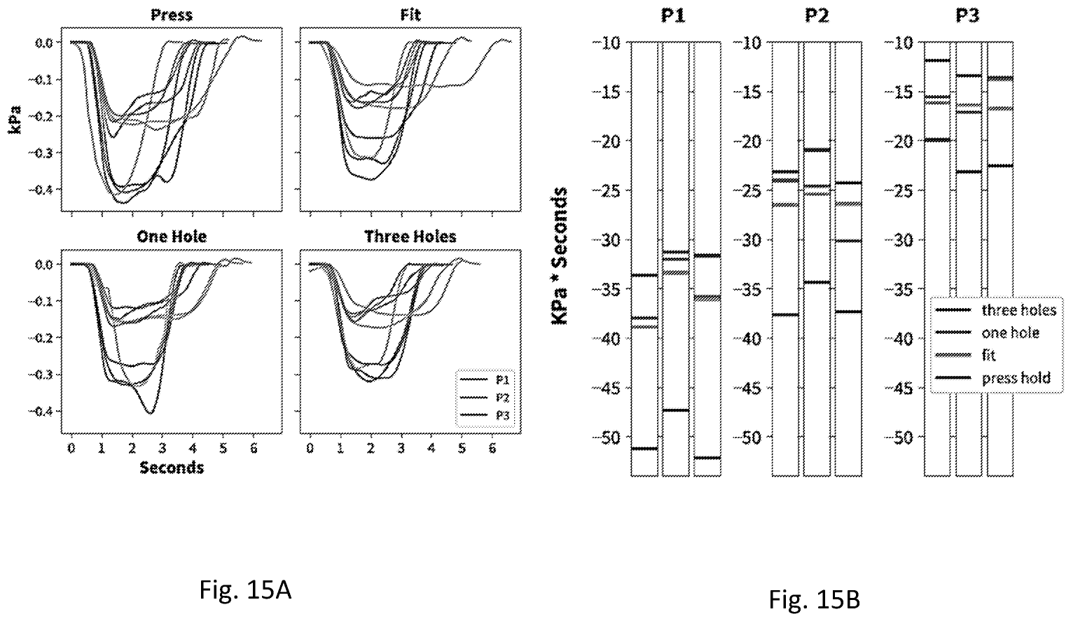

The first study was exploratory (n=3) and was conducted using FaceBit. The inventors instructed participants to perform IVCTs under three different mask-fit configurations. The configurations were self-mask fit (donned using CDC guided user-seal check), mask press (cupping one's hand and pressing the mask against the face to form a stable seal), one hole open, and three holes open. The inventors conducted three trials per participant, randomizing the order of mask configurations in each trial. FIG. 15A depicts signal from inspiratory vital capacity tests (IVCT) recorded on-board FaceBit showing filtered pressure signals for each participant (n=3) grouped by fit in accordance with an illustrative embodiment.

The pressure signals from FaceBit were collected wirelessly and analyzed in a Python notebook. The integral of the IVCT pressure signal was computed, which is proportional to total mask resistance. FIG. 15B shows integral values from initial evaluation for participants and each experiment compared to each leak configuration in accordance with an illustrative embodiment. All trials showed a larger IVCT integral during the mask-press configuration than the other configurations. Seven of nine trials measured a larger IVCT integral with the self-fit mask than a mask with a single hole. The IVCT integral was smallest in the three-hole condition in all trials.

Taking these results as promising, the inventors conducted a larger study (n=10) with a similar setup, except that pressure was measured with an external, differential pressure sensor connected via a length of tubing to the N95 mask. A differential sensor has the advantage of automatically subtracting atmospheric pressure from the measurement. Participants performed the same IVCT over five mask configurations (mask fit, mask press, one, two, and three holes). The inventors conducted three trials per participant, randomizing the order of mask configurations in each trial. Between each trial, the participant doffed and re-donned the mask. Before the experiment, all participants were presented with a CDC video on donning and doffing an N95 face mask and performing a user seal check.

Taking advantage of the increased participant count, the inventors performed a statistical analysis of the results. First, the inventors combined each participant's three trials into one by taking the mean of the IVCT integral of each mask configuration. Then, a repeated-measures analysis of variance (ANOVA) test was performed across the five mask configurations. It was found that the data violated the sphericity assumption, and so the inventors applied the Greenhouse-Geisser correction. This test yielded a corrected p-value of 6.57^{e-5}, showing a significant difference between IVCT integrals between configurations. The inventors therefore continued with a post-hoc paired T-test for each pair of mask configurations, which showed a significant difference between both the mask-press and mask-fit configurations with all other mask configurations. However, the difference between configurations with one, two, and three holes were not enough to show significance compared with each other.

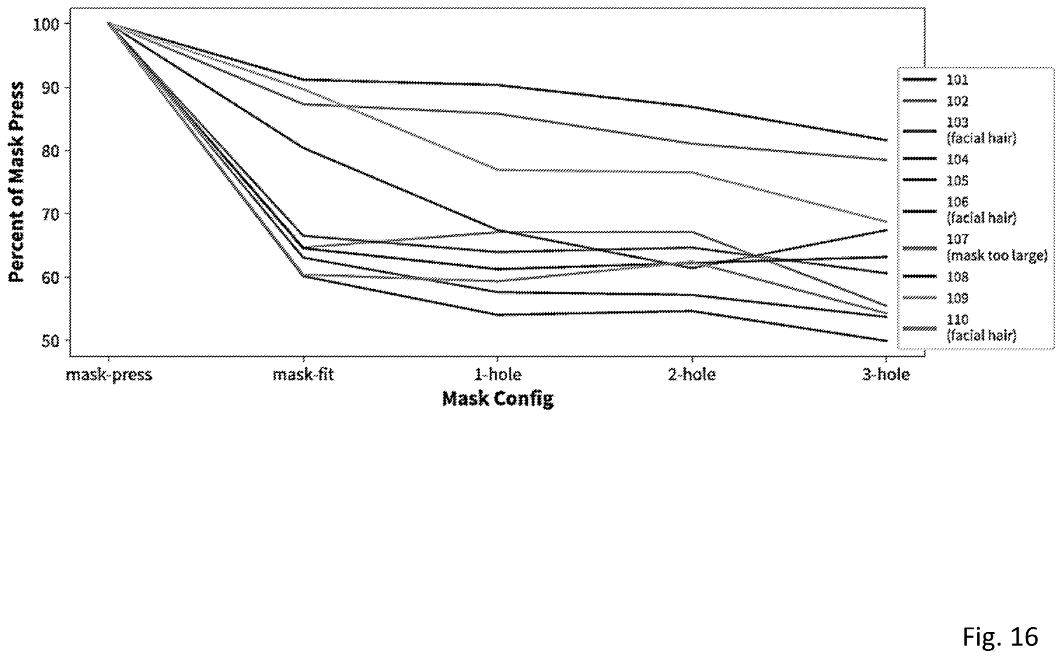

FIG. 16 depicts results of the different mask-fit configurations in accordance with an illustrative embodiment. As discussed, the integral value of five mask configurations were measured while performing a inspiratory vital capacity test (IVCT). FIG. 16 show the percent of change over the mask-press configuration which allows one to compare individual capacities side-by-side. The trend shows the integral value generally decreases as more leaks are introduced. Physiological differences of the wearers are noted in the legend. The statistical analysis of the second trial suggests this is a valid method for detecting mask leakage. These results are promising in that they show that pressure sensing used in conjunction with an easily-performed lung capacity exercise are able to quantify mask-fit in real-time.

To further test the robustness and performance of FaceBit, the inventors conducted a small scale study where three participants wore N95 masks retrofitted with a FaceBit, and performed targeted, noisy, daily life activities. By placing participants in noisy situations one can see concentrated usage of the device and find interesting confounding situations that can spark future work. The inventors made four changes for this experiment based on lessons learned during the in-lab study. First, the inventors used a new respiration rate ground truth sensor (a Vernier Respiration Belt), as the NeuLog was not capable of maintaining accurate respiration rate measurements while mobile.

Second, instead of relying on the calculated heart rate measurement from the Polar as ground truth, the inventors instead calculated heart rate from the raw EKG data of the Polar using the HeartPy Python library. In the previous experiments, it was found that the Polar calculated heart rates were potentially phase-shifted and filtering out heart rate values that were being measured, behaving much like a moving average filter.

Third, building on the lessons learned in the in-lab evaluation, the inventors modified the runtime BCG algorithm to more aggressively reject time windows where significant motion was occurring. This would necessarily reduce the number of samples one might gather, since one might reject windows where it was possible to calculate an accurate heart rate, but it is contended that this tradeoff is worthwhile since the FaceBit could be worn for twelve hours or more in a day in a clinic, or while working at a desk due to indoor masking requirements, providing ample time to gather a large amount of heart rate measurements. The sampling window was also increased: in these scenarios, FaceBit attempts to measure heart rate once per minute with a sampling window of 30 seconds.

Finally, the inventors also modified the respiratory rate algorithm to allow measurement of high respiratory rates (in the range of 30-60 BrPM) and better reject oscillations that may result from the filter. Otherwise, the setup for these experiments was similar to the first evaluation: a participant wears an N95 instrumented with Facebit along with several ground truth devices. The Vernier Respiration Belt is strapped around the chest and measures the forces associated with chest expansion during respiration, and a Polar H10 chest strap is worn under the clothes to capture EKG data from which one can calculate heart rate. Once a participant is instrumented, a study coordinator accompanies the participant (but does not interfere in any way beyond observation and data collection) on the scenarios they conduct, and uses a phone to record the ground truth values, as well as capture GPS and speed data using the Strava app.

The inventors conducted four different scenarios, ranging in length from 13 to 27 minutes. The scenarios were captured during a high heat warning (exceeding 90 degrees Fahrenheit/32 degrees Celsius), and high humidity (from 50-70%), further testing the robustness of the temperature based respiratory algorithm. The scenarios are outlined in brief: (1) Walk through town to pick up lunch. Including multiple stops on busy cross streets, waiting in line to pick up food, entry/exit from air conditioning to hot outside, and sitting at table before eating; (2) Ride on train. Including waiting at the train stop, entry and exit, and the train ride itself with multiple stops; (3) Ride in a car through a busy town, including turns, stop lights and signs, and quick accelerations/decelerations; and (4) Sit at a desk and work. This scenario was captured as a baseline, where the participant just works and types at a desk normally. Each scenario was performed once. Different participants were used for the “walk to lunch” and “work at desk” scenarios, while the same participant was used for the “ride on train” and “ride in car” scenarios.

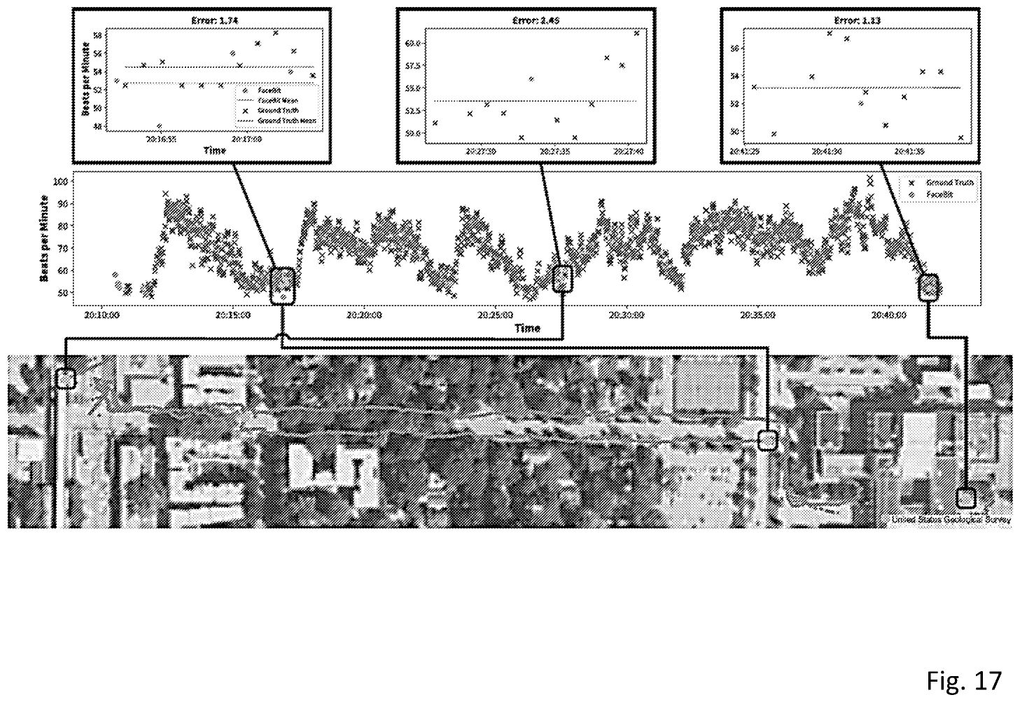

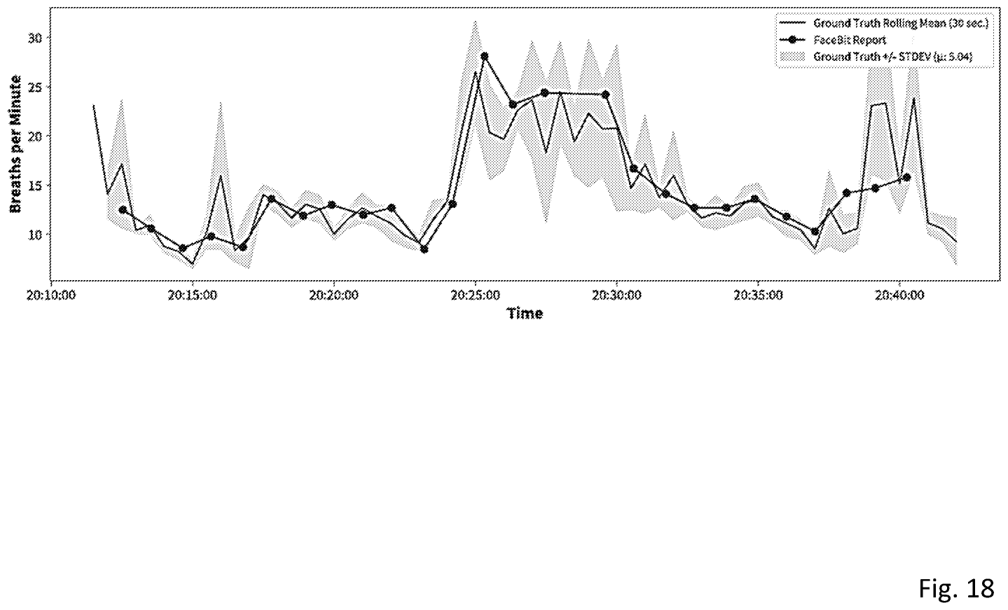

FIG. 17 depicts results from the walk to lunch scenario in accordance with an illustrative embodiment. The data shows that FaceBit is able to capture accurate heart rate at times of low-motion despite noisy conditions, and environmental factors like heat and humidity. The middle portion of FIG. 17 shows annotations where heart rate measurements were gathered by FaceBit, plotted over the ground truth heart rate where each marker is the instantaneous heart rate associated with each beat. The top portion of FIG. 17 depicts zoomed-in data-sections to highlight error, and the bottom portion is a GPS plot of the walking path with indicators of FaceBit heart rate recordings. FIG. 18 shows the FaceBit reported respiration rate plotted over the calculated rolling average of instantaneous respiration rates from the ground truth respiration belt in accordance with an illustrative embodiment. FIG. 18 also relates to the walk to lunch scenario. Similar evaluations were conducted for the other scenarios.

Overall, 2.09 beats per minute RMSE for FaceBit calculated heart rate, and a 2.80 breaths per minute RMSE for respiration were found, all in noisy scenarios. These results are very promising, demonstrating that FaceBit can be worn and used effectively in diverse, noisy situations (from heat, movement, and other diverse conditions that happen in an urban environment). The inventors are particularly encouraged that the improvements to the FaceBeat algorithm resulted in no obvious heart rate outliers, even in these challenging conditions. That said, there are also places for improvement, mainly, in balancing the need to throw out high motion periods for accuracy, and the need to gather heart rates when highly active. Indeed, on average FaceBit reported almost two heart rates per minute in the “at computer” condition, but only once every six minutes in the train condition. FIG. 17 shows that heart rate data is usually gathered when heart rate is lowest, corresponding to periods of standing still (i.e. at crosswalks, at deli-counter) and at rest (i.e. sitting at table).

Below is a discussion of how physiological differences amongst humans may confound or make it harder to preserve signal fidelity for health metrics. The data gathered from the participants in the studies, and observations made are discussed. While making a definitive statement about generalizability against human differences is impossible for any wearable device because humans are so diverse, one can draw some interesting findings from the existing data and intuition on FaceBit's function.

FaceBit is intended as a general purpose platform, and the particular metrics chosen as a demonstration of the platform (i.e., heart rate and respiration rate) are gathered from robust signals that originate from common physiological aspects of humans. Respiration rate, especially, is presumed to be robust to physiological difference as it is gathered from temperature changes in the mask caused by breath. The respiration action in the mask causes replacement of warm air with cool air brought in from outside the mask, and this signal appears to be universal in all the tests, even in participants for whom the mask was too large (n=1), who had facial hair that interfered with mask seal (n=3), and for larger persons over six foot (n=1).

Differences in the cardiovascular system that arise from height, weight, health, athleticism, and biological sex may alter the dominant frequencies, strength, and overall pacing of the BCG signal. In practice it was found that the algorithm was able to gather data from every participant in both of the evaluations, albeit with a higher degree of accuracy after the algorithmic changes. It is believed that tailoring the algorithm to an individual by adjusting filter and threshold parameters may allow for more heart rate measurements and increased accuracy.

The leak detection methodology depends on a consistent lung capacity, but it does not rely on the magnitude of the lung capacity itself. Lung capacity varies by individual, and that is why the inventors score the IVCTs as a percentage of the press-fit condition, rather than the absolute difference. The studies had a small but heterogeneous participant group across gender, size, and facial features. The results suggest the device is able to gather signals from a diverse population.

The inventors also explored the effect of mask type (N95, Surgical) and FaceBit location in the mask on the signal fidelity of the respiration and heart rates. For the experiment, each variable was isolated for both metrics of interest. The inventors collected data from a single male user with no facial hair, and in the 18-35 age range.

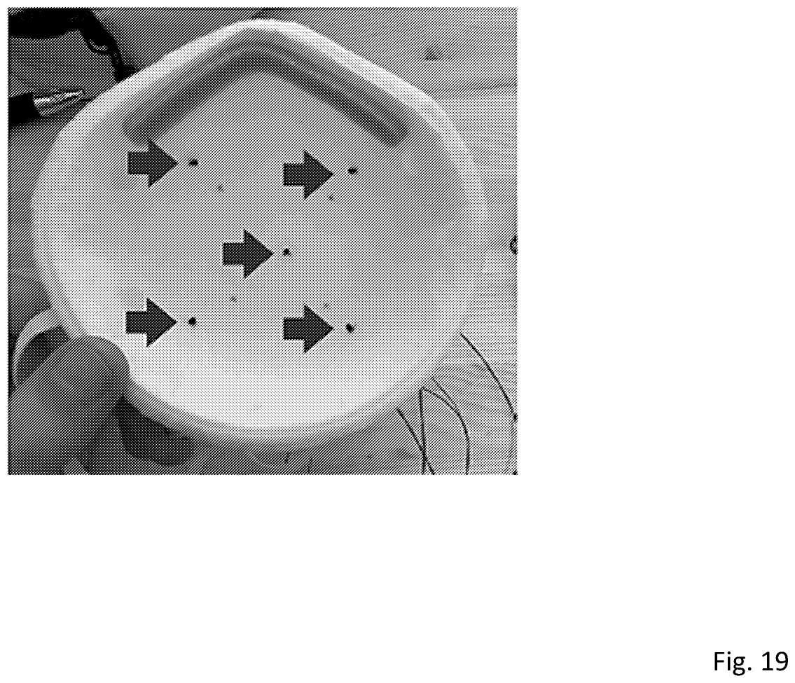

For respiration rate, since the change in temperature is used to calculate respiration, it is sought to understand the variation of temperature within masks during normal breathing. The aim is to establish whether device placement will have an effect on the respiration rate calculations. A surgical and an N95 mask were instrumented with five high precision thermistors placed in a star shape at the center and extreme ends of the mask where a FaceBit could feasibly fit without making contact with the face. FIG. 19 depicts thermistor placements in a N95 mask in accordance with an illustrative embodiment. Users then breathe into the mask regularly for 1-2 minutes, and record the simultaneous output of the thermistors, which give a continuous temperature reading.

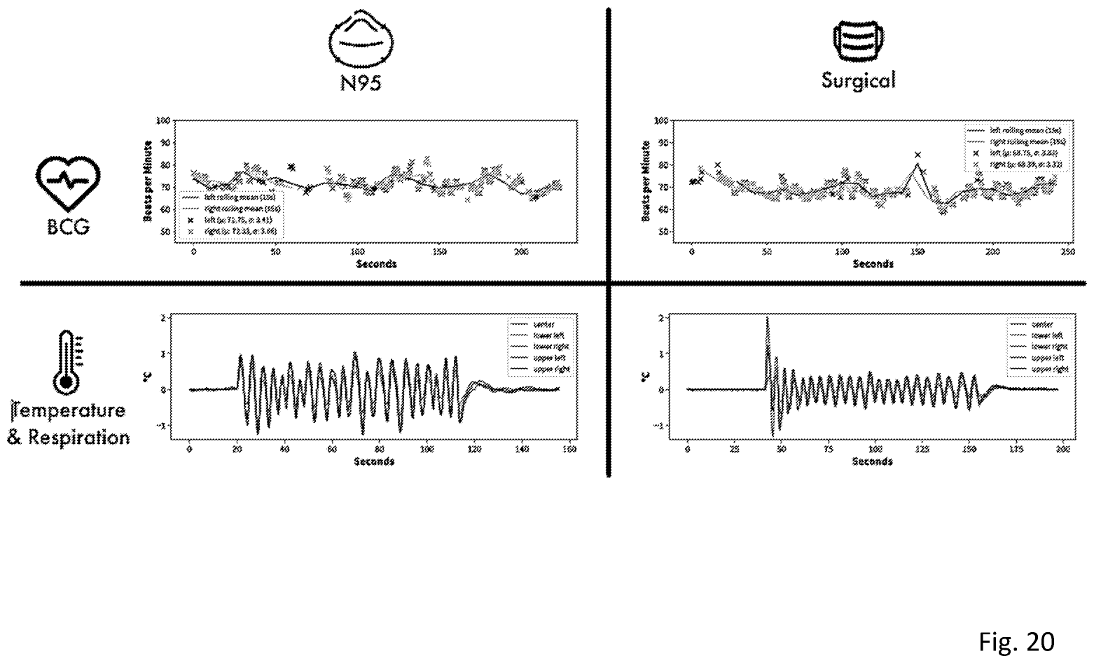

For both masks, the waveform is nearly exactly the same, with minor shifts in amplitude because of thermistor calibration offsets. After being put through the same bandpass filter as on the FaceBit, the differences shrink but do not entirely disappear. FIG. 20 shows filtered results of the thermistor analysis in accordance with an illustrative embodiment. The top portion of FIG. 20 includes the BCG signals captured for heart rate from two FaceBit devices placed on opposite sides of the mask, reporting signals with a very similar frequency content (just phase shifted). On the bottom are shown the filtered temperature signals at five placements in the mask, captured simultaneously, showing near perfect correlation. The left side shows N95 masks, and the right shows Surgical masks. This demonstrates the robustness of the signals to device location and mask type. Notably, the temperature fluctuations in the N95 mask are greater than those in the surgical mask (likely due to better trapping of air in the N95). The amplitudes of the breathing signal vary across mask locations, but the signal is clearly distinguishable throughout the mask in both cases.

Temperature change is intuitively a robust mechanism for respiration rate. The pocket of air inside the mask is small, and the volume of each breath is relatively large, therefore the volume of air inside the mask is replaced entirely with each inhalation and exhalation. If the air in the environment is cooler or (less likely) warmer than the exhaled air, these air replacements also change the temperature in the mask. Certain locations may receive a higher flow rate if the air preferentially takes a certain path out of the mask (as may be in the case of leaks in an N95 mask, or in the normal operation of a surgical mask), and therefore see higher temperature variation during breathing. In a surgical mask or small N95 mask, the sensors may be pressed against the skin in which case they receive less airflow, and therefore less temperature variation during breathing.

For heart rate, a similar experiment was conducted where two FaceBit devices are placed inside each type of mask at opposite sides. In the N95 mask, the devices are placed as far from each other as possible without being pressed against the skin. In the surgical mask, they are inset 1 inch from the left and right sides. Users don the mask and then breathe normally for 3-4 minutes, and record the heart rates reported by both devices via a JLink connection to a desktop computer. The results are shown in FIG. 20. In both mask types very similar heart rates were obtained from the two devices. Occasionally one device would lose the signal and stopped reporting heart rates while the other maintained it, however both devices resumed reporting in short order. A moving average trend placed over both scatter-plots is shown, indicating a high degree of agreement between both sensors.

This result makes sense because the ballistic force of the heart is carried through the N95 and surgical masks via their attachment to the face. Since the structures are tight to the face in both cases, and therefore rotate with the head, the rotational forces are seen throughout the masks. The BCG algorithm (and namely the combination of the gyroscopic signals across the three axes via the euclidean norm) is responsible for making the signal processing robust to device orientation.

From these experiments, one can see that the fidelity of either signal is not very sensitive to device location. This confirms the results of the two studies (in-lab and scenario based), where the devices were not carefully placed prior to data collection. In the in-lab study, the participants were asked to place the FaceBit device themselves in the center of the mask. The inventors did not modify or change the placement of the FaceBit, so eight users for which captured data on for heart rate was performed made 8 different placements. Similarly for the scenario-based study, the devices were not carefully placed in any specific location.

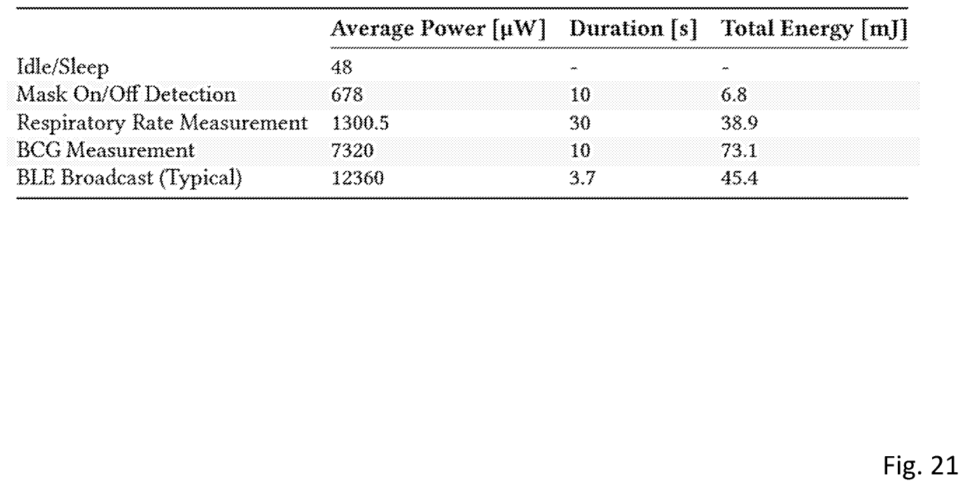

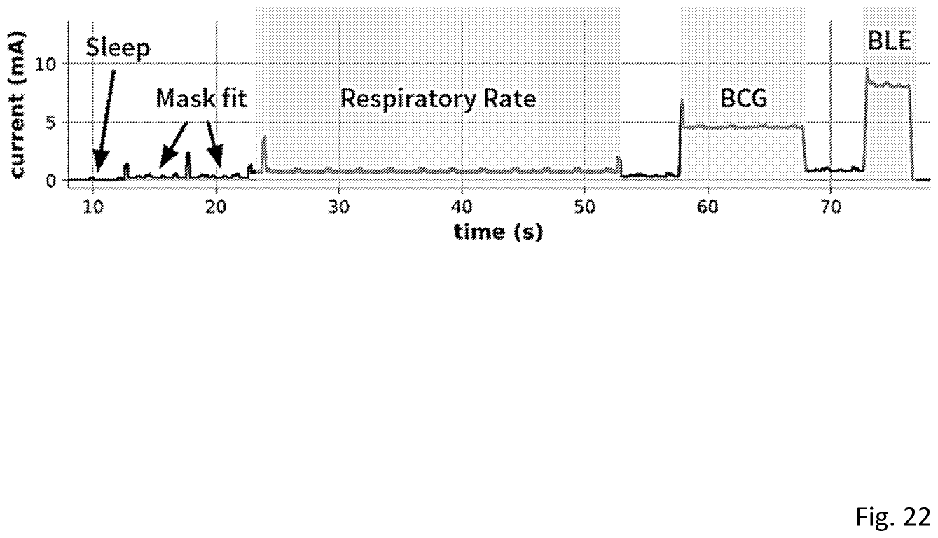

The nRF52832 is capable of a deep-sleep current consumption of 1.9 μA, but various circuitry elements (e.g. pull-down resistors) bring the sleep current of this version of the FaceBit platform up to approximately 32 μA. FIG. 21 is a table that includes a summary of the power and energy requirements of the various states that comprise the FaceBit application in accordance with an illustrative embodiment. FIG. 22 depicts an application power trace over time while calculating health metrics in accordance with an illustrative embodiment. Spikes are from current inrush due to hand resetting between tests with the test infrastructure. For the purposes of arriving at a battery life estimate, it is assumed 8 hrs/day of active on-face measurement, a moderate measurement duty cycle of one respiratory rate and heart rate every five minutes, and one BLE broadcast every five minutes. Mask On/Off status is updated every two minutes. The coin cell has a nominal capacity of 105 mWh (378 mJ), but this estimate was revised to an estimated usable capacity of 75.6 uWh (272 mJ). The inventors arrived at this number by taking into account the converter efficiency of ˜90%, and by applying an engineering factor of 80% to account for non-ideal conditions like temperature swings, self-leakage, and unused capacity below the cold-start limit. In this scenario, on battery power alone the device is expected to last for approximately eleven days.

A user burden analysis was also conducted. The FaceBit device is designed for use in many environments, but with special attention paid to clinical environments where health care workers are required to wear protective face masks throughout the workday. As part of the clinician study, a portion of the survey was devoted to the FaceBit prototype's comfort, serving as an initial user-burden analysis to drive an ongoing iterative design progress. The clinicians were provided with instructions to wear the FaceBit device with a 3D printed enclosure, and a stand-in SATURN harvester component. They then were instructed to perform a few simple exercises including adjusting the mask, moving the head from side to side and up and down, talking, and deep breathing. Participants had two magnets to attach, one for the FaceBit device, and one for the harvester below the nose.

All participants succeeded in attaching the device independently without the aid of the study coordinator. It took on average 97 seconds to follow the instructions and attach the FaceBit device to their mask. The participants were asked to give feedback on the experience, including rating the comfort of wearing a face mask with and without FaceBit attached. The average reported difference of 11 participants on comfort before and after attaching FaceBit was −3.18 on a scale of 1 to 10. One response was removed due to an error in response, where they recorded an increase in comfort which was not reflected in their open-text response.

As the survey's secondary goal, the inventors report on feedback from users to explore future design strategies. Three participants (25%) reported considering wearing FaceBit daily in its current state. Two participants reported no difference in face mask's comfort after attaching FaceBit. Additionally, 7 participants forgot to remove their FaceBit, and continuously wore it during the end-of-session discussion. The inventors consider these initial findings to be promising for the development of a consumer-ready product.

The inventors have also developed a second version of the FaceBit system. Version two of the device is planned to be similar in form to version one. In an illustrative embodiment, it includes a small, custom, printed circuit board and a partner phone application. The circuit board includes a bluetooth-enabled microcontroller, a rechargeable battery, and a variety of sensors (as described herein) that enable health and mask sensing capabilities. In one embodiment, the sensors included are an inertial measurement unit (IMU), a barometer, a temperature sensor, and a microphone. In some embodiments, the power harvesting circuitry is not included to simplify the electrical design of the device, make the device smaller, and less expensive to manufacture. In other embodiments, the power harvesting circuitry can be included as discussed herein.

The mask sensing device uses its temperature sensors and pressure sensor to infer the user's respiratory rate. These same sensors can detect when a mask is being worn. These capabilities have been validated in version one of the device. These sensors are also used to sense how well the user's mask is fitting. For example, the barometer is used to measure the integral of the pressure drop across the mask in a “well-fit” condition, and then used subsequent pressure readings to detect the presence of any leaks. The integral of the pressure drop across the mask decreases with increasing leak area for the same volume of air. This strategy can be expanded using other sensors, for example an air flow sensor to measure the speed of air exiting the mouth.

The device can be housed in a plastic enclosure with an integrated magnet that is designed to attract to another magnet mounted to a mask. This plastic enclosure may include a hydrophobic membrane that prevents water ingress while allowing the pressure inside of the enclosure to match the pressure within the mask. Additionally, there will be a second magnetic component that allows the device to be mounted to the inside of the mask, sandwiching the mask material between it and the device. This consolidation of the system allow the device to be in a smaller form factor and at a lower price. In addition to sensors, for ease of development and usability, a button for user-input (e.g., resetting the device) can be included on the plastic enclosure.

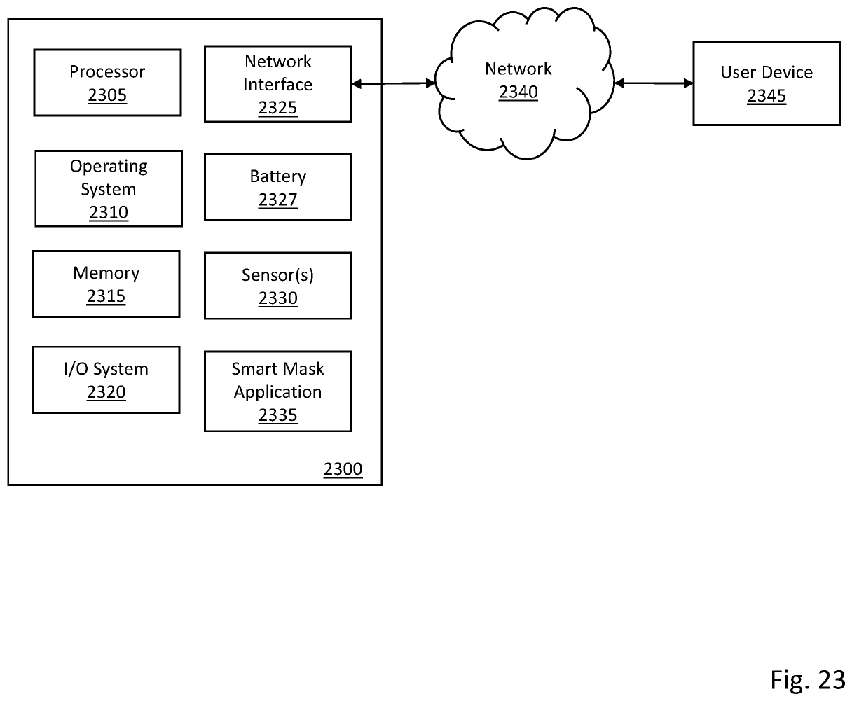

FIG. 23 is a block diagram of components included in a smart mask system in accordance with an illustrative embodiment. More specifically, FIG. 23 shows a smart mask device (FaceBit) 2300 in direct or indirect communication with a network 2340. In implementation of the smart mask device 2300 of FIG. 23, all of the different components of the device can be incorporated onto a circuit board and housed in a housing as described herein. The smart mask device 2300 includes a processor 2305, an operating system 2310, a memory 2315, an input/output (I/O) system 2320, a network interface 2325, sensor(s) 2330, and a smart mask application 2335. In alternative embodiments, the smart mask device 2300 may include fewer, additional, and/or different components. The components of the smart mask device 2300 communicate with one another via circuit board traces, one or more buses, or any other interconnect system.

The processor 2305 of the smart mask device 2300 can be in electrical communication with and used to perform any of the operations described herein, such as gathering sensed data, processing the gathered data, processing the data, sending data to a user device or other external systems, generating alerts or instructions, etc. The processor 2305 can be any type of computer processor known in the art, and can include a plurality of processors and/or a plurality of processing cores. The processor 2305 can include a controller, a microcontroller, an audio processor, a graphics processing unit, a hardware accelerator, a digital signal processor, etc. Additionally, the processor 2305 may be implemented as a complex instruction set computer processor, a reduced instruction set computer processor, an x86 instruction set computer processor, etc. The processor 2305 is used to run the operating system 2310, which can be any type of operating system.

The operating system 2310 is stored in the memory 2315, which is also used to store programs, algorithms, network and communications data, peripheral component data, the smart mask application 2335, and other operating instructions. The memory 2315 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 2320, or user interface, is the framework which enables users (and peripheral devices) to interact with the smart mask device 2300. In alternative embodiments, the I/O system 2320 can be on a remote computing device, such as the user device 2345 in the form of a phone application that communicates with the smart mask device 2300. The I/O system 2320 can include one or more keys or a keyboard, one or more buttons, a speaker, a microphone, etc. The I/O system 2320 allows the user to interact with and control the smart mask device 2300. The I/O system 2320 can also include circuitry and a bus structure to interface with and control peripheral computing components such as one or more power sources, etc.

The network interface 2325 includes transceiver circuitry (e.g., a receiver and/or a transmitter) that allows the smart mask device 2300 to transmit and receive data to/from other devices such as the user device 2345. The user device 2345 can be a cell phone, tablet, laptop computer, desktop computer, etc. The user device 2345 can also be in the form of one or more remote computing systems, servers, websites, etc. The network interface 2325 enables communication through the network 2340, which can be one or more communication networks. The network 2340 can include a cable network, a fiber network, a cellular network, a wi-fi network, a landline telephone network, a microwave network, a satellite network, etc. The network interface 2325 also includes circuitry to allow device-to-device communication such as near field communication (NFC), Bluetooth® communication, etc.

The smart mask device 2300 also includes a battery 2327 to power the various components of the device. Any type of battery 2327 can be used. In an illustrative embodiment, the battery 2327 is a rechargeable battery that the user is able to periodically charge, either wirelessly or through a charging cable. In an alternative embodiment, the smart mask device 2300 can also include one or more energy harvesting devices, as described herein to automatically charge the battery while the device is in use.

The smart mask device 2300 also includes one or more sensor(s) 2330. The one or more sensor(s) 2330 can include a temperature sensor, a barometer or other pressure sensor, an inertial measurement unit (e.g., one or more gyroscopes and/or one or more accelerometers), an air quality sensor, a magnetometer, a flow rate sensor to monitor breathing, etc. As discussed herein, the one or more sensor(s) 2330 can be used to detect whether the mask is being worn, an amount of time that the mask has been worn, a respiration rate of the wearer, a heart rate of the wearer, air quality, etc.

The smart mask application 2335 can include software and algorithms (e.g., in the form of computer-readable instructions) which, upon activation or execution by the processor 2305, performs any of the various operations described herein such as activating sensors, recording sensed data, processing the sensed data to determine information regarding mask usage, processing the sensed data to determine physiological information regarding the wearer, processing the sensed data to determine air quality, transmitting sensed data and/or processed information to a remote device, generating alerts based on mask usage or monitored physiological parameters, receiving instructions from a remote user device, etc. The smart mask application 2335 can utilize the processor 2305 and/or the memory 2315 as discussed above.

It seems increasingly likely that worldwide face mask usage is not going away. Even before the COVID-19 pandemic, health care workers regularly used PPE, and many countries' populations are regular users of face masks or other forms of personal protective equipment to deal with air quality or prevent viral infections. With tens of billions of face masks manufactured a month, poor air quality, and respiratory infections on the rise, there is an unprecedented need for intelligent and active PPE. This disclosure presents the FaceBit platform as both a novel wearable health device and a new platform enabling a community of diverse researchers in the emerging space of smart face masks. FaceBit balances a large design space, offering rich sensing with a small form factor and long battery lifetime.

In some embodiments, FaceBit attempts to make the vision of smart PPE more sustainable by taking advantage of energy harvested from the environment and incorporating energy-efficient operation. With innovations in low power design, signal processing for health metrics, energy harvesting, and form factors, the platform offers a foundation for future health research and a jumping off point for new design of smart face masks. While the COVID-19 crisis is the primary motivation for this subject matter, the contributions to building smart personal protective equipment go beyond the pandemic, and provide new ways to think about how to protect vulnerable populations. Due to the low user burden of the platform, it is believed FaceBit will catalyze mobile health sensing despite the pandemic and assist with long term, high adherence studies within ubiquitous and mobile computing, health and human behavior, and novel on-body sensing.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A smart face mask device comprising:
a housing that is designed to mount to a personal protective face mask;
a memory within the housing that is configured to store sensor data;
one or more sensors configured to generate the sensor data during use of the personal protective mask; and
a processor operatively coupled to the memory and configured to:
process the sensor data to determine information regarding a user of the personal protective mask; and
transmit the information regarding the user to a remote application.

2. The device of claim 1, wherein the one or more sensors include a pressure sensor such that the sensor data includes sensed pressure values within the personal protective face mask.

3. The device of claim 2, wherein the processor is configured to process the sensed pressure values to assess mask fit of the personal protective face mask.

4. The device of claim 3, wherein the processor is configured to determine an integral of a pressure drop across the personal protective face mask to assess the mask fit.

5. The device of claim 3, wherein the processor is configured to generate an alert responsive to a determination that the mask fit of the personal protective face mask does not satisfy a mask fit threshold.

6. The device of claim 1, wherein the one or more sensors include a temperature sensor such that the sensor data includes temperature values within the personal protective face mask.

7. The device of claim 6, wherein the processor processes the temperature values to determine a respiration rate of the user of the personal protective face mask.

8. The device of claim 7, wherein the processor generates an alert responsive to a determination that the respiration rate is not within a threshold range of respiration rates.

9. The device of claim 1, wherein the one or more sensors include an inertial measurement unit such that the sensor data includes information regarding movement of the personal protective face mask.

10. The device of claim 9, wherein the processor processes the information regarding movement of the personal protective face mask to determine a heartbeat of the user of the personal protective face mask.

11. The device of claim 10, wherein the processor generates an alert responsive to a determination that the heartbeat of the user falls outside of a threshold range of heartbeats.

12. The device of claim 1, wherein the one or more sensors include a pressure sensor, and wherein the processor is configured to determine whether the mask is being worn based on pressure readings from the pressure sensor.

13. The device of claim 12, wherein the processor is configured to determine an amount of time that the mask has been worn based on the pressure readings from the pressure sensor.

14. The device of claim 13, wherein the processor is configured to generate an alert to change masks responsive to a determination that the amount of time that the mask has been worn exceeds a threshold value.

15. The device of claim 1, further comprising a first magnet mounted to the housing, wherein the first magnet is configured to mate with a second magnet on the personal protective face mask to secure the device to the personal protective face mask.

16. A method of monitoring a face mask, the method comprising:
obtaining sensor data from one or more sensors mounted to a housing that is secured to a personal protective face mask;
storing the sensor data in a memory that is within the housing;
processing, by a processor operatively coupled to the memory, the sensor data to determine information regarding a user of the personal protective mask; and
transmitting the information regarding the user to a remote application.

17. The method of claim 16, wherein the one or more sensors include a pressure sensor such that obtaining the sensor data includes obtaining pressure values within the personal protective face mask.

18. The method of claim 17, further comprising processing, by the processor, the obtained pressure values to assess mask fit of the personal protective face mask.

19. The method of claim 16, wherein the one or more sensors include a temperature sensor such that obtaining the sensor data includes obtaining temperature values within the personal protective face mask.

20. The method of claim 16, further comprising processing, by the processor, the obtained temperature values to determine a respiration rate of the user of the personal protective face mask.
